(19) United States
(12) Patent Application Publication
(10) Pub. No.: US 2019/0240017 A1
(43) Pub. Date: Aug. 8, 2019

(54) THREADING DEVICES, ELONGATED MEMBERS, AND METHODS OF MANUFACTURE AND USE THEREOF

(71) Applicant: Ergosuture Corp., Arlington, MA (US)

(72) Inventors: Luis Jose Almodovar, Santa Maria, PR (US); Marcio Scorsin, Luxembourg City (LU)

(21) Appl. No.: 16/238,922

(22) Filed: Jan. 3, 2019

Related U.S. Application Data
(60) Provisional application No. 62/729,970, filed on Sep. 11, 2018.
(60) Provisional application No. 62/613,381, filed on Jan. 3, 2018.

(51) Int. Cl. A61F 2/24; A61B 17/06; A61B 17/062; A61B 17/04

(52) U.S. Cl. CPC A61F 2/2445; A61F 2/2466; A61B 17/0469; A61B 17/062; A61B 17/06166

(57) ABSTRACT

This disclosure discloses various elongated members that are segmentally adjustable. Further, this disclosure discloses various threading devices that can secure various toggles and various locks to various surfaces, whether animate or inanimate. Additionally, this disclosure discloses various techniques of using such elongated members and such threading devices.

### CROSS-REFERENCE TO RELATED APPLICATIONS

The present application claims the benefit of U.S. Provisional Patent Application No. 62/729,970 filed 11 Sep. 2018, and U.S. Provisional Patent Application No. 62/613,381 filed 3 Jan. 2018, each of which is herein incorporated by reference for all purposes.

### BACKGROUND

A human heart has a mitral valve with an annulus. The mitral valve may be impaired with a regurgitation condition where the mitral valve is not able to coapt properly within the annulus. The regurgitation condition may be treated by an annuloplasty procedure through an open heart surgery. Since the open heart surgery is risky, some patients do not want the open heart surgery or are not suitable for the open heart surgery.

In order to deal with such limitations, the annuloplasty procedure has been adapted for use with a catheter. This adaptation is commonly known as a transcatheter annuloplasty. The transcatheter annuloplasty involves an annuloplasty ring being implanted about the mitral valve on the annulus through a catheter. When the annuloplasty ring is implanted about the mitral valve on the annulus, the annuloplasty ring facilitates proper coaptation within the annulus and thereby treats the regurgitation condition. Although the surgical annuloplasty ring is widely used, the transcatheter annuloplasty ring is problematic for several reasons.

First, the annuloplasty ring is often based on purse-string adjustable technique, while implanted about the mitral valve on the annulus to reduce its size. As such, this form of adjustment is problematic because anatomically changing the mitral valve may affect at least some functioning of the human heart. Second, the transcatheter annuloplasty ring does not reshape the annulus. Rather, the annuloplasty ring reduces at least some dilatation of the annulus by producing a circling as a flexible band ring. Third, the annuloplasty ring often fails to correct some forms of the regurgitation condition (e.g., functional mitral regurgitation). Fourth, the annuloplasty ring is secured about the mitral valve to the annulus with a plurality of helical anchors. Therefore, since the helical anchors are invasive and are reputed to disengage, this form of implanting the annuloplasty ring about the mitral valve on the annulus may lead to more trauma or various complications of at least the human heart. Fifth, some transcatheter annuloplasty rings avoid closing the mitral valve via the purse-string adjustable technique and can be modulated in a segmental manner. However, these rings are also fixed to the annulus with the helical anchors and have a design that is not low-profile.

### SUMMARY

In an embodiment, a device comprises: a sleeve including a plurality of segments that are adjustable independent of each other when the sleeve is secured onto a surface.

In an embodiment, a device comprises: a first tube; a second tube extending within the first tube; a needle including a shape memory material, wherein the needle is switchable between a non-default shape and a default shape based on the shape memory material, wherein the needle has the non-default shape when the needle extends within the second tube, wherein the needle has the default shape when the needle extends outside the second tube; a rod extending within the needle; a toggle extending within the needle, wherein the rod engages the toggle; a thread extending within the first tube, wherein the thread is coupled to the toggle; a lock extending within the first tube, wherein the lock is coupled to the thread; a foot extending within the first tube, wherein the foot is retractable out of the first tube; and a cutting edge extending within the first tube, wherein the cutting edge is sufficiently sharp to cut the thread above the lock.

In an embodiment, a method comprises: laying a sleeve onto a surface, wherein the sleeve includes a plurality of segments; securing the sleeve onto the surface from within the sleeve; and adjusting the segments independent of each other from outside the sleeve.

In an embodiment, a method comprises: piercing a surface at a first location with a needle, wherein the needle includes a toggle and a thread, wherein the toggle is coupled to the thread; extending the needle below the surface such that the needle pierces the surface at a second location; outputting the toggle about the second location above the surface such that the thread extends below the surface from the toggle to the first location; positioning a lock about the first location such that the thread spans between the lock and the toggle; and cutting the thread above the lock.

In an embodiment, a device comprises: an elongated member including a plurality of segments that are adjustable independent of each other when the elongated member is secured onto a surface of a valve of a heart.

### DESCRIPTION OF DRAWINGS

FIG. 1 shows a cross-section profile diagram of an embodiment of a threading device in accordance with this disclosure.

FIG. 2 shows a perspective diagram of an embodiment of a threading device in accordance with this disclosure.

FIGS. 3A-3F show\s a plurality of profile diagrams of an embodiment of a technique for using a threading device in accordance with this disclosure.

FIGS. 4A-4C show a plurality of profile diagrams of an embodiment of a technique for using a threading device in accordance with this disclosure.

FIGS. 5A-5D show a plurality of perspective diagrams of an embodiment of a threading device in accordance with this disclosure.

FIGS. 6A-6B show a plurality of perspective diagrams of an embodiment of an elongated member in accordance with this disclosure.

FIGS. 7A-7B shows a plurality of profile diagrams of an embodiment of a tube hosting a needle with a shape memory material in accordance with this disclosure.

FIGS. 8A-8C show a plurality of perspective diagrams of a technique for adjusting an elongated member in accordance with this disclosure.

FIGS. 9A-9D show a plurality of perspective diagrams of a technique for adjusting an elongated member in accordance with this disclosure.

FIGS. 10A-10C show a plurality of perspective diagrams of a technique for adjusting an elongated member in accordance with this disclosure.

FIGS. 11A-11C show a plurality of perspective diagrams of a technique for adjusting an elongated member in accordance with this disclosure.

FIG. 12 shows a perspective diagram of a technique for adjusting an elongated member in accordance with this disclosure.

FIG. 13 shows a perspective diagram of a technique for adjusting an elongated member in accordance with this disclosure.

FIG. 14 shows a plurality of diagrams of a front-biting suturing assembly for attaching an annuloplasty ring to a cardiac tissue in accordance with this disclosure.

FIGS. 15A-15B show a plurality of diagrams of a front-biting suturing assembly for attaching an annuloplasty ring to a cardiac tissue in accordance with this disclosure.

FIGS. 16A-16B show a plurality of diagrams of a front-biting suturing assembly for attaching an annuloplasty ring to a cardiac tissue in accordance with this disclosure.

FIGS. 17A-17B show a plurality of diagrams of a front-biting suturing assembly for attaching an annuloplasty ring to a cardiac tissue in accordance with this disclosure.

FIGS. 18A-18B show a plurality of diagrams of a front-biting suturing assembly for attaching an annuloplasty ring to a cardiac tissue in accordance with this disclosure

FIG. 19 shows a diagram of a continuous driving system for transcatheter/minimally invasive suturing in accordance with this disclosure.

FIG. 20 shows a diagram of a continuous driving system for transcatheter/minimally invasive suturing in accordance with this disclosure.

FIG. 21 shows a diagram of a continuous driving system for transcatheter/minimally invasive suturing in accordance with this disclosure.

FIG. 22 shows a diagram of a continuous driving system for transcatheter/minimally invasive suturing in accordance with this disclosure.

FIG. 23 shows a diagram of a continuous driving system for transcatheter/minimally invasive suturing in accordance with this disclosure.

FIG. 24 shows a diagram of a continuous driving system for transcatheter/minimally invasive suturing in accordance with this disclosure.

FIG. 25 shows a diagram of a continuous driving system for transcatheter/minimally invasive suturing in accordance with this disclosure.

FIGS. 26A-26B show a plurality of diagrams of a continuous driving system for transcatheter/minimally invasive suturing in accordance with this disclosure.

FIGS. 27A-27G show a plurality of diagrams of a continuous driving system for transcatheter/minimally invasive suturing in accordance with this disclosure.

FIG. 28 shows a diagram of a continuous driving system for transcatheter/minimally invasive suturing in accordance with this disclosure.

FIG. 29 shows a diagram of a continuous driving system for transcatheter/minimally invasive suturing in accordance with this disclosure.

FIG. 30 shows a diagram of a continuous driving system for transcatheter/minimally invasive suturing in accordance with this disclosure.

FIG. 31 shows a plurality of diagrams of a continuous driving system for transcatheter/minimally invasive suturing in accordance with this disclosure.

FIG. 32 shows a plurality of diagrams of a continuous driving system for transcatheter/minimally invasive suturing in accordance with this disclosure.

FIG. 33 shows a diagram of a continuous driving system for transcatheter/minimally invasive suturing in accordance with this disclosure.

FIG. 34 shows a plurality of diagrams of a continuous driving system for transcatheter/minimally invasive suturing in accordance with this disclosure.

FIG. 35 shows a plurality of diagrams of a continuous driving system for transcatheter/minimally invasive suturing in accordance with this disclosure.

FIG. 36 shows a diagram of a continuous driving system for transcatheter/minimally invasive suturing in accordance with this disclosure.

FIG. 37 shows a plurality of diagrams of a continuous driving system for transcatheter/minimally invasive suturing in accordance with this disclosure.

FIG. 38 shows a diagram of a continuous driving system for transcatheter/minimally invasive suturing in accordance with this disclosure.

FIG. 39 shows a plurality of diagrams of a continuous driving system for transcatheter/minimally invasive suturing in accordance with this disclosure.

FIG. 40 shows a diagram of a continuous driving system for transcatheter/minimally invasive suturing in accordance with this disclosure.

FIGS. 41A-41K show a plurality of diagrams of a continuous driving system for transcatheter/minimally invasive suturing in accordance with this disclosure.

FIGS. 42-43 show a plurality of diagrams of a plurality of embodiments of entering and exiting a threading device and an elongated member in accordance with this disclosure.

FIGS. 44A-44B, 45A-45E, 46A-46C, and 47 show a plurality of diagrams of a plurality of embodiments of an elongated member hosting a threading device in accordance with this disclosure.

FIGS. 48, 49, 50A-50B, 51A-51F, 52A-52B, and 53A-53B show a plurality of diagrams of a securing clip and a plurality of embodiments of a technique of adjusting an elongated member in accordance with this disclosure.

FIG. 54 shows a flowchart of a process for threading in accordance with this disclosure.

FIG. 55 shows a flowchart of a process for coupling an elongated member to a surface in accordance with this disclosure.

FIG. 56 shows a flowchart of a process for adjusting an elongated member in accordance with this disclosure.

### DETAILED DESCRIPTION OF PREFERRED EMBODIMENTS

Generally, this disclosure discloses various elongated members that are segmentally adjustable. Further, this disclosure discloses various threading devices that can secure various toggles and various locks to various surfaces, whether animate or inanimate. Additionally, this disclosure discloses various techniques of using such elongated members and such threading devices. Note that this disclosure may be embodied in many different forms and should not be construed as necessarily being limited to various embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected,” or “coupled” to another element, then the element can be directly on, connected, or coupled to another element or intervening elements can be present, including indirect or direct variants. In contrast, when an element is referred to as being “directly connected” or “directly coupled” to another element, then there are no intervening elements present.

As used herein, various singular forms “a,” “an” and “the” are intended to include various plural forms as well, unless specific context clearly indicates otherwise.

As used herein, various presence verbs “comprises,” “includes” or “comprising,” “including” when used in this specification, specify a presence of stated features, integers, steps, operations, elements, or components, but do not preclude the presence or addition of one or more other features, integers, steps, operations, elements, components, or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

As used herein, a term “or others,” “combination”, “combinatory,” or “combinations thereof” refers to all permutations and combinations of listed items preceding that term. For example, “A, B, C, or combinations thereof' is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. Skilled artisans understand that typically there is no limit on number of items or terms in any combination, unless otherwise apparent from the context.

As used herein, unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized or overly formal sense unless expressly so defined herein.

As used herein, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” or “substantially” refers to a +/−10% variation from a nominal value/term. Such variation is always included in any given value/term provided herein, whether or not such variation is specifically referred thereto.

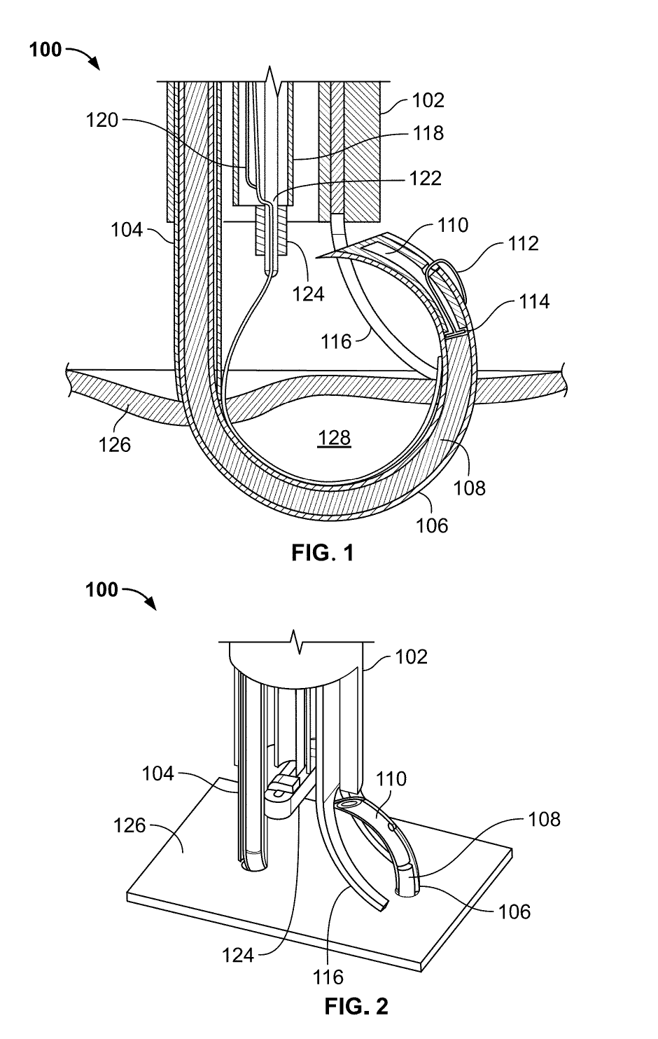

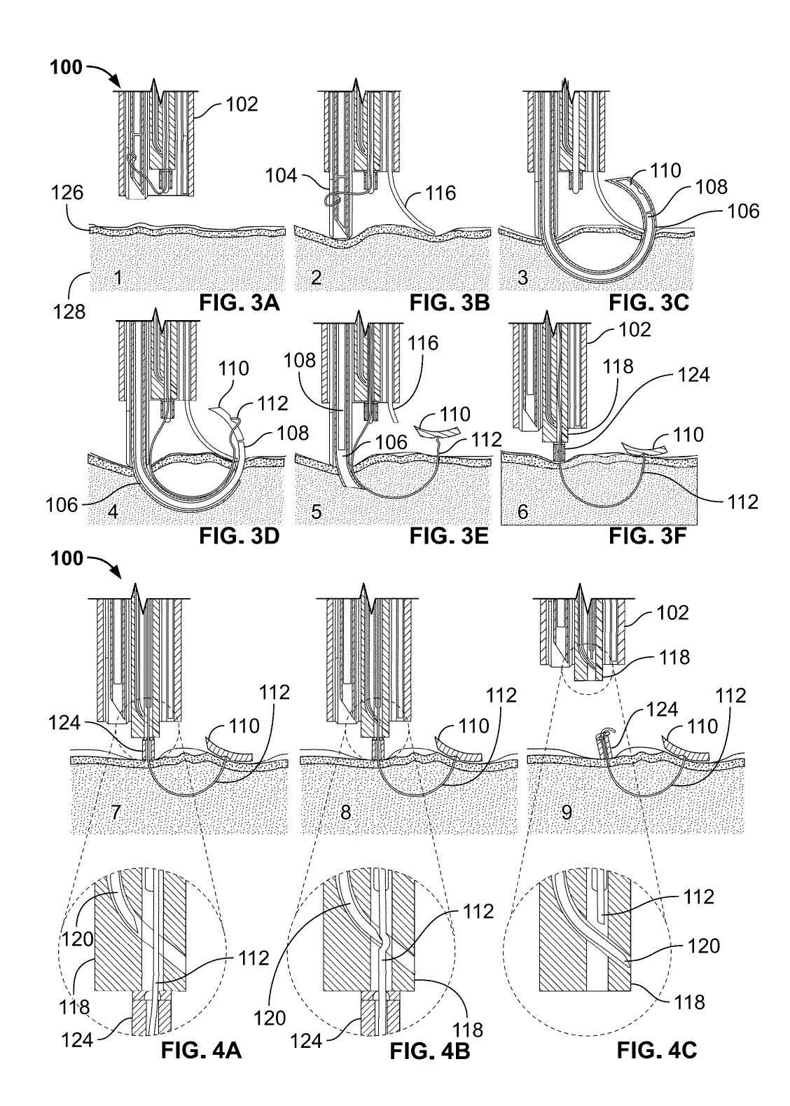

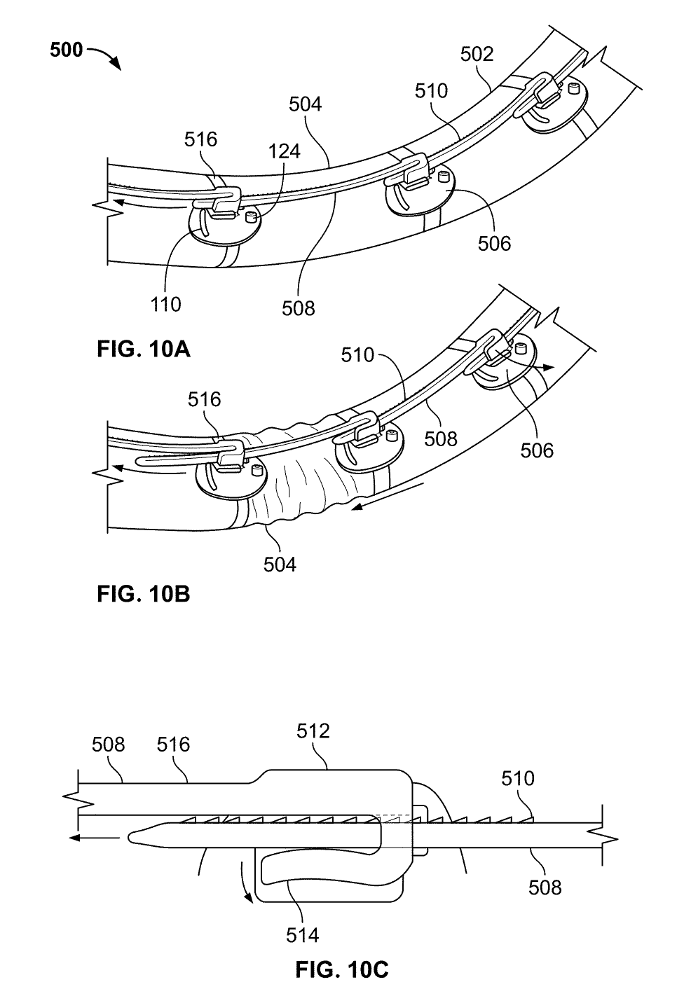

FIG. 1 shows a profile diagram of an embodiment of a threading device in accordance with this disclosure. FIG. 2 shows a perspective diagram of an embodiment of a threading device in accordance with this disclosure. FIGS. 7A-7B shows a plurality of profile diagrams of an embodiment of a tube hosting a needle with a shape memory material in accordance with this disclosure. In particular, a threading device 100 includes a first tube 102, a second tube 104, a needle 106, a rod 108, a toggle 110, a thread 112, a knot 114, a foot 116, a third tube 118, a cutting edge 120, a spacer 122, and a lock 124. The threading device 100 is used relative to a layer 126 and a volume 128.

The first tube 102 can be embodied as a catheter. The first tube 102 can include plastic, silicon, metal, rubber, ceramic, glass, leather, fabric, or other suitable materials. The first tube 102 is solid, but can be perforated. The first tube 102 can be rigid, semi-rigid, elastic, resilient, or flexible. For example, the first tube 102 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The first tube 102 can have a cross-section that is closed-shaped (e.g., O-shape, D-shape, 0-shape, square, rectangle, triangle, polygon) or open-shaped (e.g. U-shape, C-shape, V-shape), whether symmetrical or asymmetrical. The first tube 102 has an open end portion.

The second tube 104 extends within the first tube 102. The second tube 104 can telescope within the first tube 102. The second tube 104 can extend out of the first tube 102 (through the open end portion of the first tube 102) or retract into the first tube 102 (through the open end portion of the first tube 102). The second tube 104 can be embodied as a catheter. The second tube 104 can include plastic, silicon, metal, rubber, ceramic, glass, leather, fabric, or other suitable materials. The second tube 104 is solid, but can be perforated. The second tube 104 can be rigid, semi-rigid, elastic, resilient, or flexible. For example, the second tube 104 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The second tube 104 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical. The second tube 104 includes an open end portion.

The needle 106 extends within the second tube 104. The needle 106 can telescope within the second tube 104. The needle 106 can extend out of the second tube 104 (through the open end portion of the second tube 104) or retract into the second tube 104 (through the open end portion of the second tube 104). The needle 106 is solid, but can be perforated. The needle 106 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical. The needle 106 has a leading tip portion that is sufficiently sharp or speared to puncture through the layer 126 and the volume 128 such that the leading tip can lead through the layer 126 and the volume 128 when external to the second tube 104. The leading tip portion is open and can be straight, beveled, chamfered, or bull-nosed.

As also shown in FIGS. 7A-7B, the needle 106 includes a shape memory material (e.g., shape memory polymer, shape memory alloy, copper-aluminum-nickel alloy, nickel-titanium alloy, polyurethane). As such, the needle 106 is switchable between a non-default shape and a default shape based on the shape memory material. Therefore, the needle 106 has the non-default shape when the needle 106 extends within the second tube 104. The needle 106 has the default shape when the needle 106 extends outside the second tube 104. Note that the needle 106 is rectilinear when the needle 106 extends within the second tube 104 while the needle 106 is in the non-default shape. However, when the needle 106 is output from the second tube 104, the needle 106 deforms into the default shape, which is shown to be arcuate-shaped or crescent-shaped, although other shapes are possible. For example, the needle 106 can deform about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). Note that the needle 106 can also avoid the shape memory material and include plastic, silicon, metal, rubber, ceramic, glass, leather, fabric, or other suitable materials.

The rod 108 extends within the needle 106. The rod 108 can telescope within the needle 106. The rod 108 can extend out of the needle 106 (through the leading tip portion) or retract into the needle 106 (through the leading tip portion). The rod 108 can include plastic, silicon, metal, rubber, ceramic, glass, leather, fabric, or other suitable materials. The rod 108 is solid, but can be perforated. The rod 108 is internally dense, but can be hollow. The rod 108 can be rigid, semi-rigid, elastic, resilient, or flexible. For example, the rod 108 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The rod 108 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The toggle 110 extends within the needle 106 such that the rod 108 engages the toggle 110. The toggle 110 can travel within the needle 108 based on the rod 108 pushing the toggle 110. The toggle 110 can include plastic, silicon, metal, rubber, ceramic, glass, leather, fabric, or other suitable materials. The toggle 110 is solid, but can be perforated. The toggle 110 is internally dense, but can be hollow. The toggle 110 can be rigid, semi-rigid, elastic, resilient, or flexible. For example, the toggle 110 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The toggle 110 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The thread 112 can be a suture, which can be non-absorbable or absorbable of various gauges. The thread 112 can include silk, cotton, fabric, nylon, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, or other natural or artificial materials. The thread 112 is solid, but can be perforated. The thread 112 is internally dense, but can be hollow. The thread 112 can be rigid, semi-rigid, elastic, resilient, or flexible. For example, the thread 112 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The thread 112 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical. The thread 112 is coupled to the toggle 110 via the knot 114 being positioned internal to the toggle 110, although the knot 114 can be positioned external to the toggle 110. Although the knot 114 is T-shaped, the knot 114 can be shaped differently (e.g., spherical, oval, cuboid, cube, bow). Note that the thread 112 runs from the toggle 110, while looping about the needle 106 and extending along the needle 106 below the layer 126 and through the volume 128 past the second tube 104 and extending into the first tube 102, as further explained below. As shown in FIG. 1, the thread 112 runs in a U-shape, a C-shape, a sickle-shape, or a crescent-shape.

The foot 116 extends within the first tube 102. The foot 116 can retract out of the first tube 102. The foot 116 can extend within a tube (e.g., catheter) that extends with the first tube 102 and the foot 116 can retract from this tube as well. When retracted, the foot 116 can engage at least one of the thread 112, the needle 106, or the layer 126. The foot 116 can include plastic, silicon, metal, rubber, ceramic, glass, leather, fabric, or other suitable materials. The foot 116 is solid, but can be perforated. The foot 116 is internally dense, but can be hollow. The foot 116 can be rigid, semi-rigid, elastic, resilient, or flexible. For example, the foot 116 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The foot 116 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The third tube 118 extends within the first tube 102 such that the third tube 118 is positioned between the second tube 104 and the foot 116 within the first tube 102. The third tube 118 can telescope within the first tube 102. The third tube 118 can extend out of the first tube 102 (through the open end portion of the first tube 102) or retract into the first tube 102 (through the open end portion of the first tube 102). The third tube 118 can be embodied as a catheter. The third tube 118 can include plastic, silicon, metal, rubber, ceramic, glass, leather, fabric, or other suitable materials. The third tube 118 is solid, but can be perforated. The third tube 118 can be rigid, semi-rigid, elastic, resilient, or flexible. For example, the third tube 118 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The third tube 118 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical. The third tube 118 includes an open end portion.

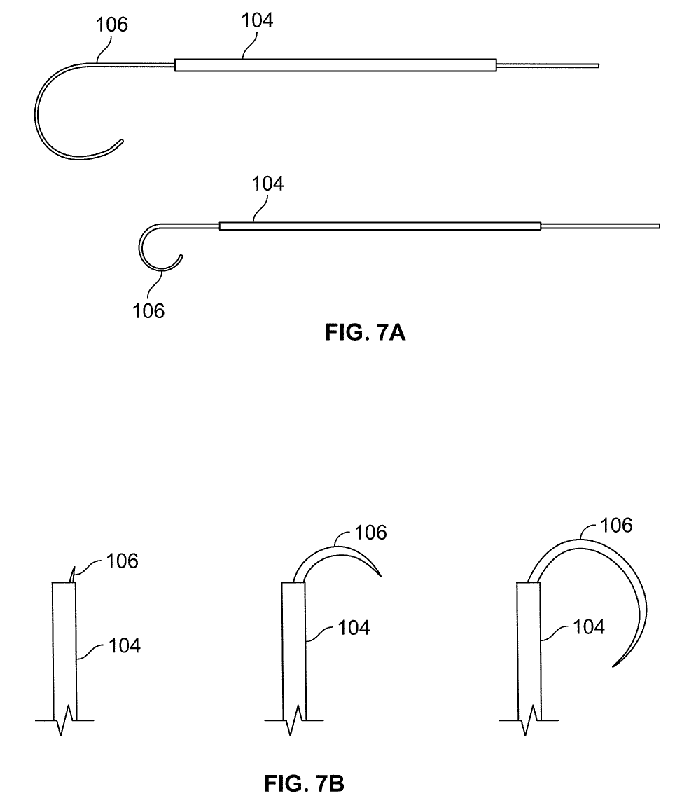

The cutting edge 120 (e.g., cutter, blade, knife, razor, sword) extends within the first tube 102 such that the cutting edge 120 is positioned between the thread 112 and at least one of the second tube 104, the needle 106, or the rod 108. The cutting edge 120 extends within the third tube 118 such that the cutting edge 120 is positioned between the thread 112 and at least one of the second tube 104, the needle 106, or the rod 108. The cutting edge 120 can have a normal shape, a trailing-point shape, a drop-point shape, a clip-point shape, a sheepsfoot shape, a wharncliffe shape, a spey point shape, a leaf shape, a spear point shape, a needle point shape, a kris or flame-bladed shape, a hawkbill shape, a chisel point shape, an ulu shape, a rotary shape, an O-shape, a U-shape, or other shapes. The cutting edge 120 is sufficiently sharp to cut the thread 112. As shown in FIGS. 1 and 4, the third tube 118 has a longitudinal length and the cutting edge 120 is configured to move lateral (e.g., side-to-side) to the longitudinal length. Therefore, the cutting edge 120 can cut the thread 112 during this lateral movement. Note that although the cutting edge 120 is employed, other ways of breaking, disassembling, or separating the thread 112 can be used, whether additionally or alternatively, such as a chemical compound, a heat source, a cold source, a laser source, or others. Further, the thread 112 can include a plurality of perforated lateral portions (like perforated toilet paper) and thereby detached by pressure onto the perforated lateral portions.

The cutting edge 120 can telescope within the third tube 118. The cutting edge 120 can extend out of the third tube 118 (out of the open end of the third tube portion) or retract into the third tube 118 (out of the open end of the third tube portion). The cutting edge 120 can include plastic, silicon, metal, rubber, ceramic, glass, leather, fabric, a shape memory material, or other suitable materials. The cutting edge 120 is solid, but can be perforated. The cutting edge 120 can be rigid, semi-rigid, elastic, resilient, or flexible. For example, the cutting edge 120 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The cutting edge 120 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The spacer 122 is positioned within the first tube 102. The spacer 122 is positioned within the third tube 118 between the cutting edge 120 and the lock 124. The spacer forms sufficient space to enable the cutting edge 120 to cut the thread above the lock 124, while enabling the thread 112 to remain coupled (e.g., knotted, attached, secured, fastened, mated, interlocked, adhered) to the lock 124. Although the spacer 122 is shown offset relative to the lock 124, the spacer 124 can be absent or non-offset relative to the lock 124. The spacer 112 can prevent the cutting edge 120 from cutting the thread 112 until the spacer 112 is moved (e.g., retracted) such that the cutting edge 120 can cut the thread 112. The spacer 112 can engage (e.g., contact) the cutting edge 120. The spacer 122 can include plastic, silicon, metal, rubber, ceramic, glass, leather, fabric, or other suitable materials. The spacer 122 is solid, but can be perforated. The spacer 122 is internally dense, but can be hollow. The spacer 122 can be rigid, semi-rigid, elastic, resilient, or flexible. For example, the spacer 122 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The spacer 122 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The lock 124 extends within the first tube 102 such that the lock 124 is positioned between the foot 116 and at least one of the second tube 104, the needle 106, or the rod 108. The lock 124 extends out of the first tube 102 such that the lock 124 is positioned between the foot 116 and at least one of the second tube 104, the needle 106, or the rod 108. The lock 124 is hosted via the third tube 118 above the layer 126 such that the layer 126 is positioned between the lock 124 and the volume 128. Note that the thread 112 runs from the toggle 110, while looping about the needle 106 and extending along the needle 106 below the layer 126 and through the volume 128 past the second tube 104 and extending through the lock 124 and into the first tube 102 and the third tube 118 past spacer 122 and the cutting edge 120. As shown in FIG. 1, the thread 112 runs in a U-shape, a C-shape, a sickle-shape, or a crescent-shape. As shown in FIG. 4, the cutting edge 120 cuts the thread 112 above the lock 124 and above the spacer 122. The lock 124 is coupled to the thread 112 (e.g., knotting, attaching, securing, fastening, pressure, mating, interlocking, adhering, magnetizing). Note that the cutting edge 120 is sufficiently sharp to cut the thread 112 above the lock 124, as shown in FIG. 4.

The layer 126 can include can include silk, cotton, fabric, fabric pledget or pad or strip or band or sleeve, nylon, suture, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, absorbable material, non-absorbable material, an animate tissue, an inanimate tissue, or other natural or artificial materials. The animate tissue can include a mammalian tissue, an animal tissue, a plant tissue, a bird tissue, a fish tissue, a reptile tissue, a human tissue, or others. The animate tissue can include a connective tissue, a muscular tissue, a nervous tissue, an epithelial tissue, a bone tissue, a cartilage tissue, an organ tissue, or others. The organ tissue can be include in a heart tissue, a brain tissue, a liver tissue, a stomach tissue, an esophagus issue, a trachea tissue, a large or small intestine tissue, a pancreatic tissue, a lung tissue, a trachea tissue, a diaphragm tissue, a kidney tissue, a bladder tissue, a urethra tissue, a gland tissue, a reproductive organ tissue, a spleen tissue, a skin tissue, an eye tissue, an ear tissue, a nose tissue, a mouth tissue, an artery tissue, a vein tissue, or others. The heart tissue can include a valve tissue, an atrium tissue (left, right), a ventricle tissue (left, right), or others. The valve tissue can include a pulmonary valve tissue, a tricuspid valve tissue, an aortic valve tissue, a mitral valve tissue, or others. The layer 126 can be solid or perforated. The layer 126 is internally dense, but can be hollow. The layer 126 can be rigid, semi-rigid, elastic, resilient, or flexible. For example, the layer 126 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The layer 126 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

The volume 128 can include an inanimate volume or an animate volume. The inanimate volume can include silk, cotton, fabric, nylon, suture, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, absorbable material, non-absorbable material, or other natural or artificial materials. The animate volume can include a tissue, such as an a mammalian tissue, an animal tissue, a plant tissue, a bird tissue, a fish tissue, a reptile tissue, a human tissue, or others. The tissue can include a connective tissue, a muscular tissue, a nervous tissue, an epithelial tissue, a bone tissue, a cartilage tissue, an organ tissue, or others. The organ tissue can be include in a heart tissue, a brain tissue, a liver tissue, a stomach tissue, an esophagus issue, a trachea tissue, a large or small intestine tissue, a pancreatic tissue, a lung tissue, a trachea tissue, a diaphragm tissue, a kidney tissue, a bladder tissue, a urethra tissue, a gland tissue, a reproductive organ tissue, a spleen tissue, a skin tissue, an eye tissue, an ear tissue, a nose tissue, a mouth tissue, an artery tissue, a vein tissue, or others. The heart tissue can include an intra-cardiac tissue, a valve tissue, an atrium tissue (left, right), a ventricle tissue (left, right), or others. The valve tissue can include a pulmonary valve tissue, a tricuspid valve tissue, an aortic valve tissue, a mitral valve tissue, or others.

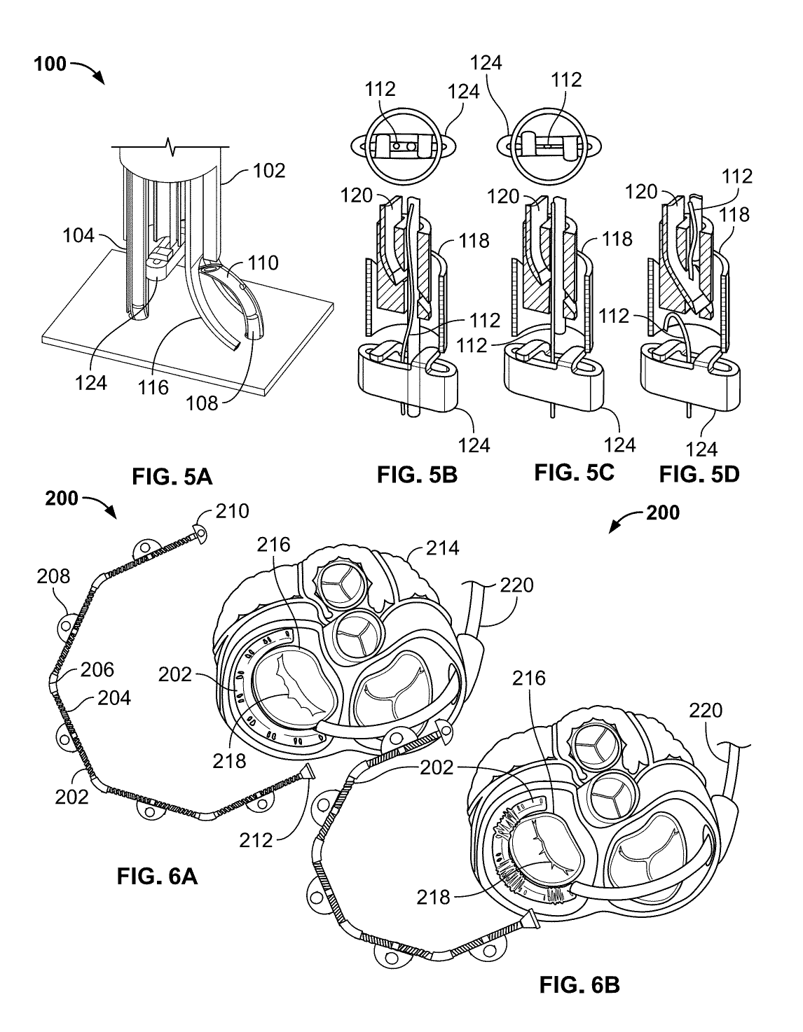

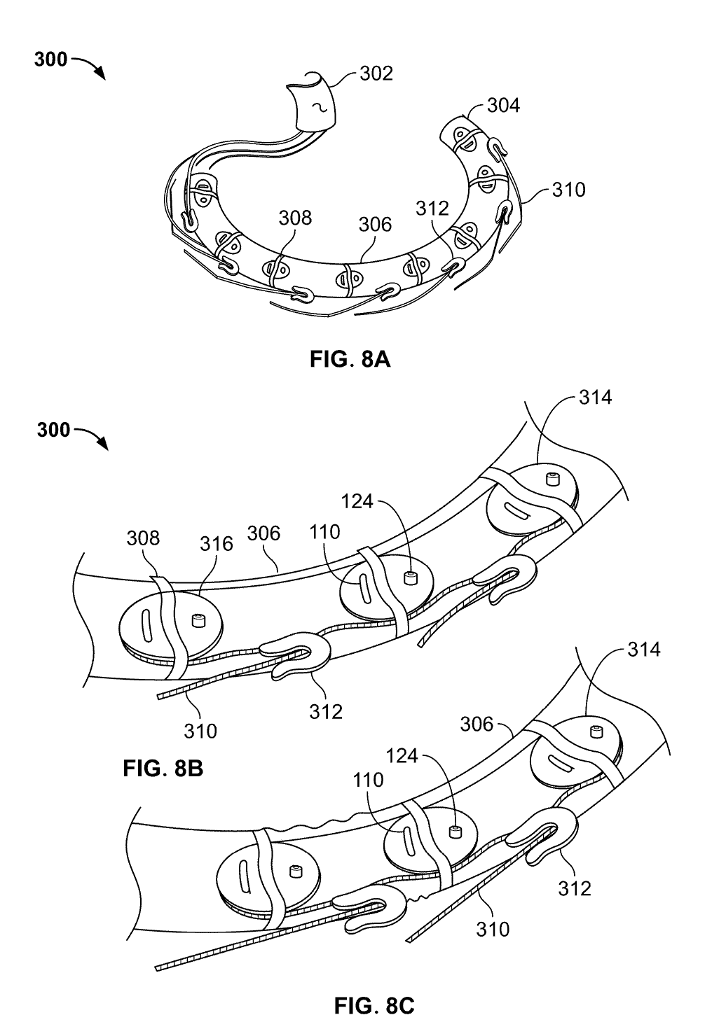

FIGS. 3A-3F and 4A-4C show a plurality of profile diagrams of an embodiment of a technique for using a threading device in accordance with this disclosure. FIGS. 5A-5D show a perspective diagram of an embodiment of a threading device in accordance with this disclosure. In particular, as per profile diagram 1, the threading device 100 is positioned such that the open end portion of the first tube 102 opposes the layer 126, the open end portion of the first tube 102 faces the layer 126, and the layer 126 is positioned between the first tube 102 and the volume 128.

As per FIG. 3B, the second tube 104 is moved (e.g., telescoped) out of the first tube 104 and the foot 116 is moved out of the first tube 102 such that the second tube 104 contacts the layer 126 and the foot contacts the layer 126. The second tube 104 contains the needle 106 hosting the toggle 110 coupled to the thread 112. The foot 116 bends laterally relative to the second tube 104 based on contacting the layer 126. Note that the second tube 104 and the foot 116 descend such that the layer 126 and the volume 128 are indented.

As per FIG. 3C, the needle 106 is output (e.g., telescoped) from the second tube 104 such that the needle 106 pierces the layer 126 at a first location, travels through the volume 128 below the layer 126, and pierces the layer 126 at a second location such that the toggle 110 is positioned above the layer 126. The needle 106 travels in an arcuate, crescent, U-shape, or C-shape when output from the second tube 104 based on the shape memory material being allowed to deform into the default shape. Note that the needle 106 travels through the volume 128 while carrying the toggle 110 and the thread 112 to the second location.

As per FIG. 3D, the needle 106 retracts (e.g., telescoped) into the second tube 104 as the rod 108 is advanced or held in place, thereby outputting (e.g., ejecting) the toggle 110. Note that the thread 112 runs from the toggle 110, while looping about the needle 106 and from the second location extending along the needle 106 below the layer 126 and through the volume 128 out from the first location past the second tube 104 and extending through the lock 124 and into the first tube 102 and the third tube 118 past spacer 122 and the cutting edge 120. The thread 112 runs in a U-shape, a C-shape, a sickle-shape, or a crescent-shape.

As per FIG. 3E, the rod 108 retracts (e.g., telescoped) into the needle 106, the needle 106 retracts (e.g., telescoped) into the second tube 104, and the foot 116 retracts into the first tube 102 (e.g., telescoped). The toggle 110 is about to land on the surface 126 while the thread 112 runs from the toggle 110 through the second location into the volume 128 out from the first location into the lock 124 and into the third tube 118 and into the first tube 102.

As per FIG. 3F, the first tube 102 is moved towards the layer 128, although the first tube 102 can remain stationary. The third tube 118 is moved (e.g., telescoped) out of the first tube 102 such that the lock 124 contacts the layer 126, such that the lock 124 extends between the third tube 118 and the layer 126, and such that the layer 126 extends between the lock 124 and the volume 128. The thread 112 is pulled, which can be taut, and the lock 124 is advanced and locked in position.

As per FIG. 4A, the lock 124 is locked in position based on retracting the spacer 122, while the toggle 110 rests on the layer 128 and the thread 112 runs from the toggle 110 at the second location through the volume 128 below the layer 126 through the lock 124 into the third tube 118.

As per FIG. 4B, the cutting edge 120 is advanced to cut the thread 112. Note that, as explained above, other ways of breaking, disassembling, or separating the thread 112 can be used, whether additionally or alternatively, such as a chemical compound, a heat source, a cold source, a laser source, or others. Further, the thread 112 can be include a plurality of perforated lateral portions (like perforated toilet paper) and thereby detached by pressure onto the perforated lateral portions.

As per FIG. 4C, the thread 120 is cut. Therefore, the thread 112 spans between the toggle 110 and the lock 124, which running through the volume 128 between the first location and the second location. This structure now holds the layer 126 to the volume 128.

In some embodiments, as per FIGS. 1-5D, a suture is delivered through an annulus of a mitral valve using a hollow super-elastic Nitinol needle (e.g., the needle 106) that is heat-set to adopt a curved profile. When retracted into a tubular housing (e.g., the second tube 104), the Nitinol needle is forced straight, (1) reducing the diameter of the catheter sheath required to house Nitinol needle and (2) allowing the Nitinol needle to be actuated by a simple push/pull element. As the Nitinol needle is extended from the tubular housing, the Nitinol needle cuts a curved path through the tissue (e.g., the volume 128), re-emerging at a distance close to the arc diameter. This also includes (1) a toggle (e.g., the toggle 110) to anchor the distal end of the suture (e.g., the thread 112) to the fabric pledget (e.g., the layer 126), (2) a toggle pusher (e.g., the rod 106) to eject the toggle, (3) a suture lock (e.g., the lock 124), to clamp the proximal end of the suture when the lock spacer (e.g., the spacer 122) is removed, and (4) a cutter (e.g., the cutting edge 120) to trim the suture to length once the suture lock has been secured. For example, (1) the threading device 100 approximates to a suturing site, (2) the needle housing and retractable foot descend, indenting the fabric and the tissue, (3) the hollow needle is inserted, carrying the toggle and suture to the second anchor point, (4) the needle retracts as the toggle pusher is advanced or held rigid, thereby ejecting the toggle, (5) the needle and toggle pusher retract, leaving the suture embedded in the tissue, (6) the suture is pulled tight, and the suture lock is advanced and locked in position, (7) the suture lock is locked in position by retracting the lock spacer, (8) the cutter is advanced to sever the suture, and (9) the toggle now securely holds the fabric pledget to the tissue. This can be beneficial since transcatheter sutures secure an annuloplasty implant to closely replicate surgical annuloplasty, are outcome comparable to surgical rings, and effectively avoid or minimize invasive anchors (less trauma). When used with an elongated member (e.g., annuloplasty ring), then the elongated member reduces or reshapes annulus dilatation through the transcatheter route, while the elongated member is capable to reshape the native annulus according to its size and shape as efficient as semi-rigid surgical rings, and can be addressed at least to mitral and tricuspid valves of the human heart. Further, the elongated member provides a reduction or decrease in size of the mitral annulus and preserves its natural shape by the elongated member having separate but interlinked contractable/adjustable segments. This structure translates to an ability for selectively contracting certain parts of the annulus more than others.

In some embodiments, based on FIGS. 1-5B, a transcatheter suturing system can be configured to allow close apposition of tissue edges or structures (e.g., paravalvular leak correction, patent foramen ovale closure, valve leaflet approximation).

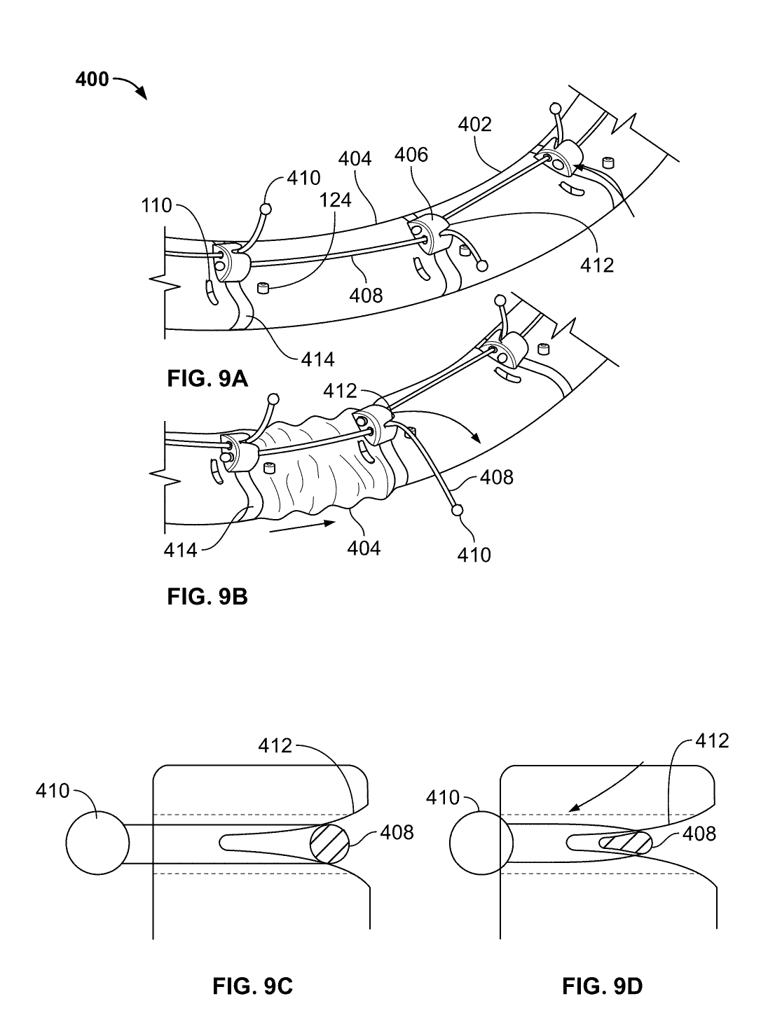

FIGS. 6A-6B show a perspective diagram of an embodiment of an elongated member in accordance with this disclosure. In particular, an elongated member 200 includes a body 202, a plurality of segments 204, a plurality of joints 206, a plurality of eyelets 208, a first longitudinal end portion device 210, and a second end longitudinal end portion device 212. The elongated member 200 can be delivered via a catheter 220. The elongated member 200 can be used in a context of various surfaces (inanimate and animate) and various volumes (inanimate and animate), as disclosed herein. As shown in FIG. 6A, one of such surfaces and one of such volumes can be a human heart 214 having a mitral valve with an annulus 216 and impaired with a medical condition or disease. The annulus 216 has a leaflet seam 218. For example, the medical condition or disease can include a regurgitation condition, such as a mitral regurgitation, whether primary or secondary/functional or a tricuspid valve regurgitation.

As shown in FIG. 6A, the elongated member 200 can be embodied as a Segmentally Adjustable Transcatheter Annuloplasty Ring (SATAR) device. The SATAR device includes the segments 204 that are interconnected at the joints 206 and can be individually, selectively, and independently contracted or expanded some more than others, as shown in FIG. 6B, in order to optimize a reshaping of the annulus 216, while preserving the D-shape of the annulus 216. Each of the segments 204 has a mechanism that, when actuated by a flexible shaft inside the SATAR device, shortens a length of that respective segment or each segment as desired. As a flexible shaft moves along the SATAR device, this process may be repeated at each segment until a desired configuration is reached.

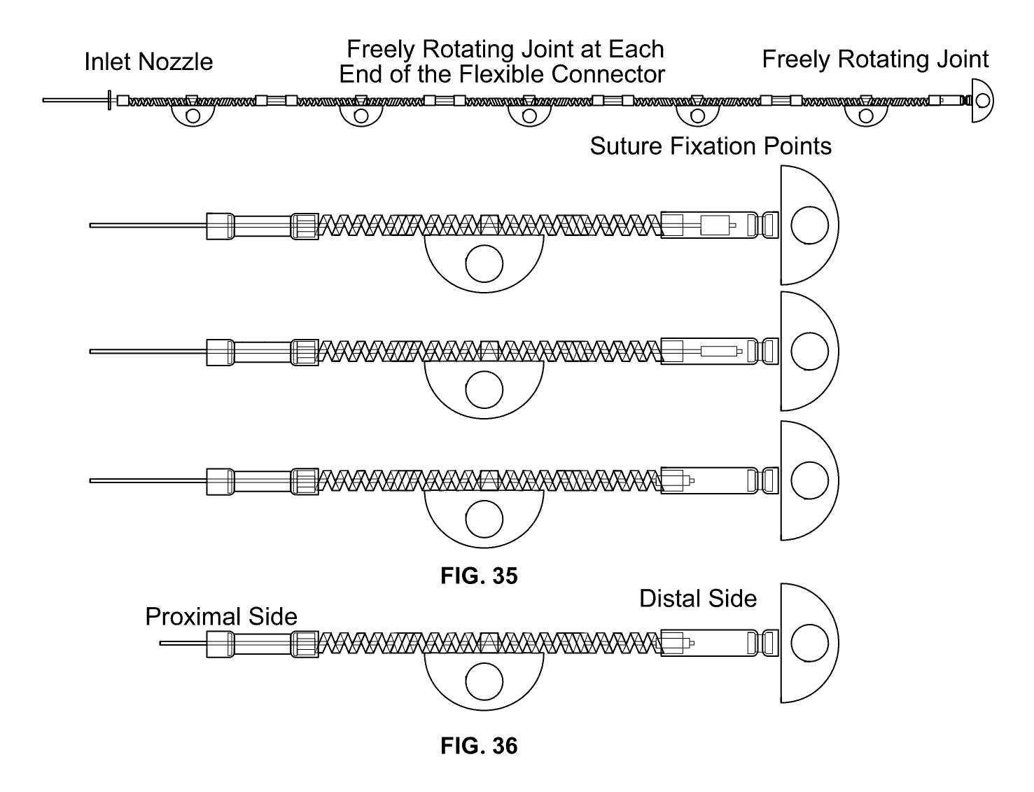

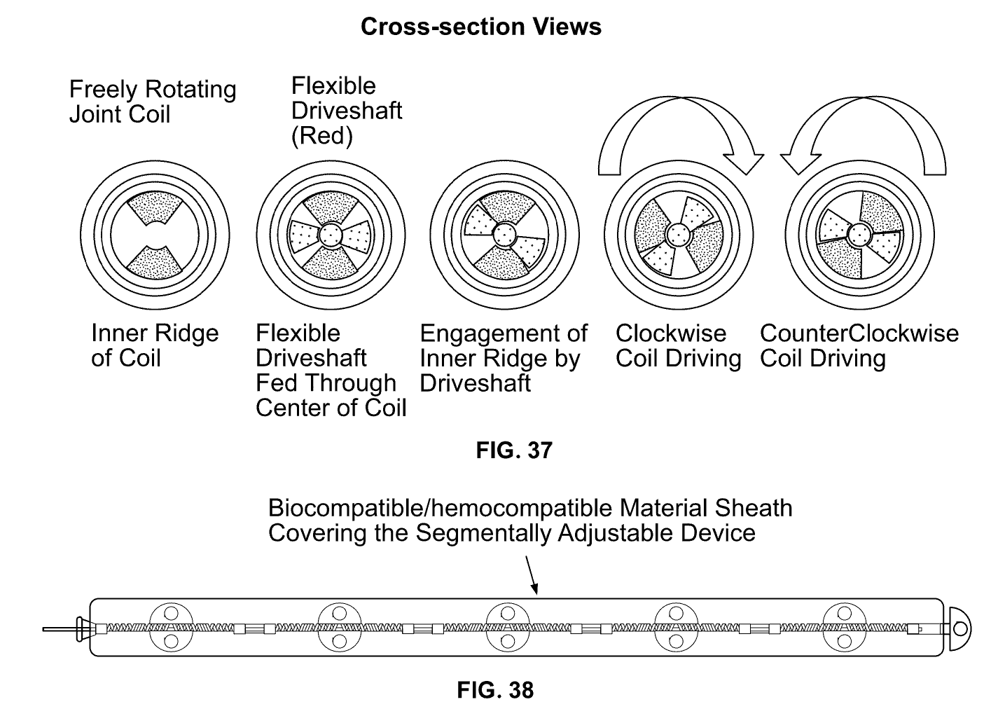

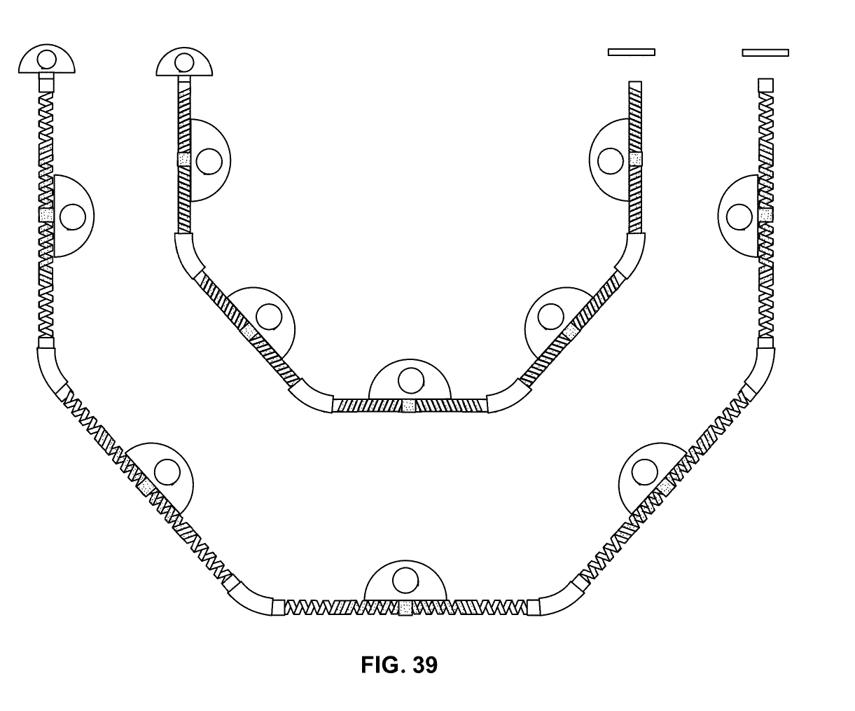

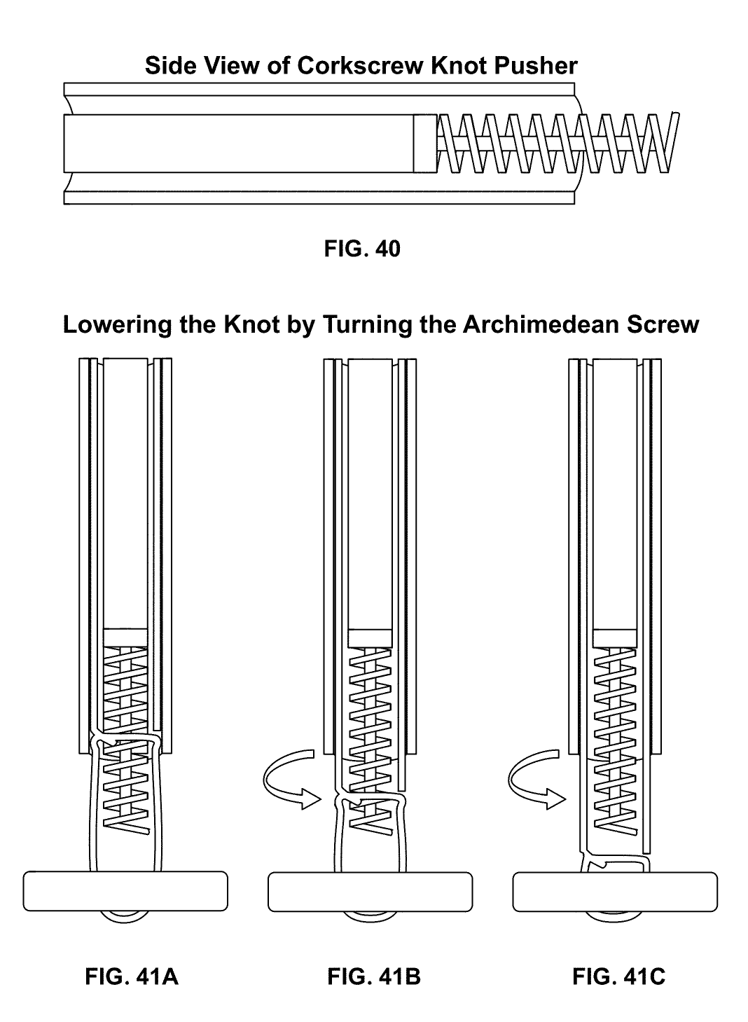

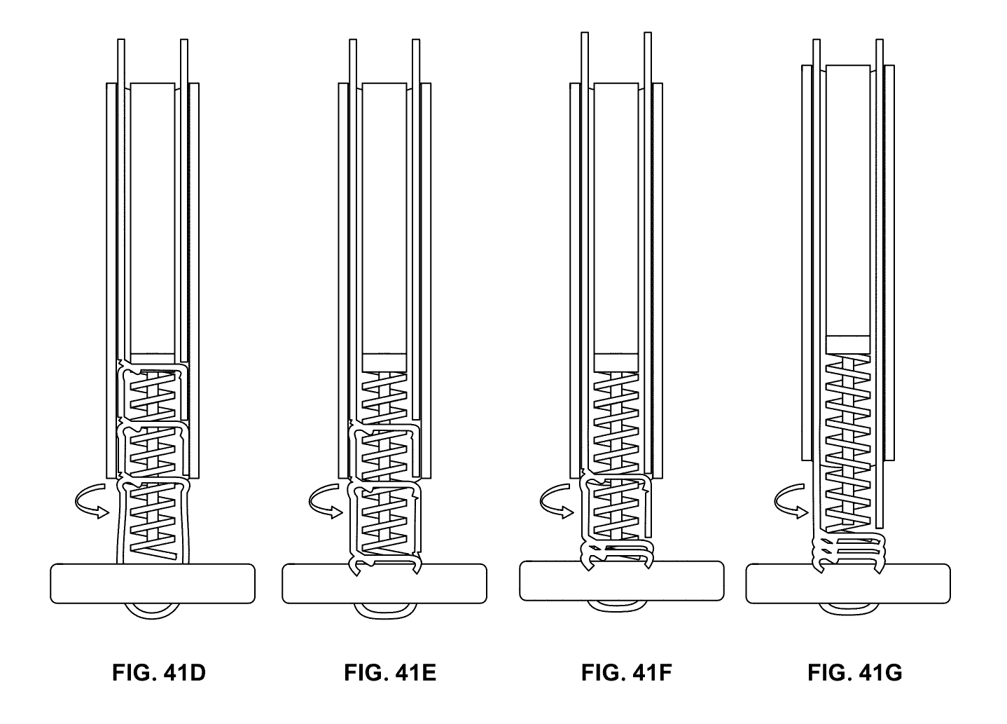

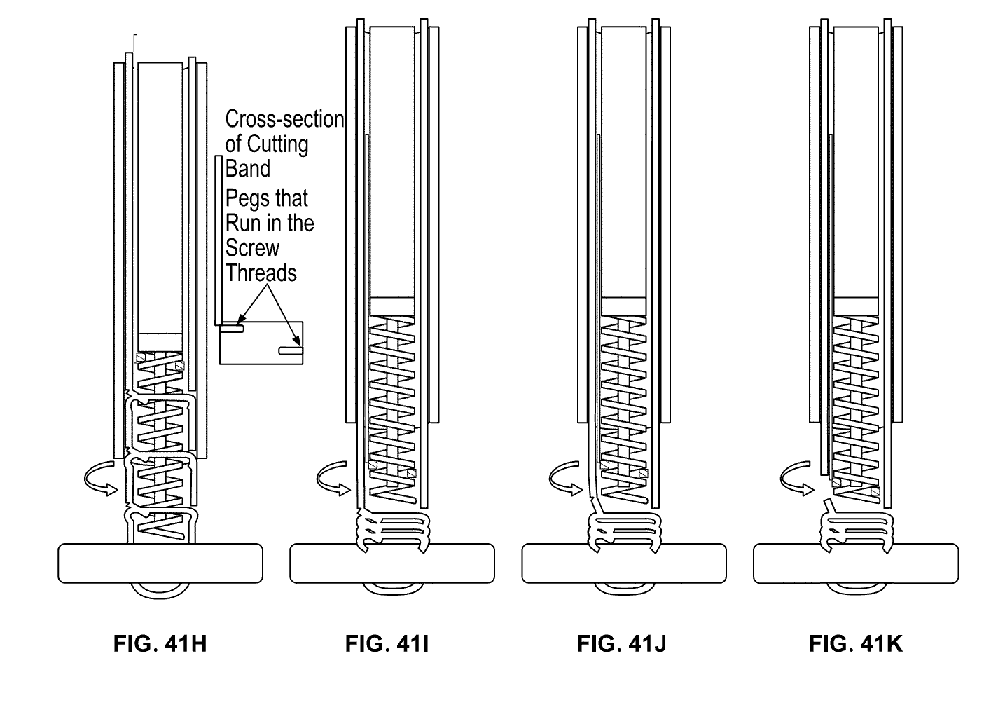

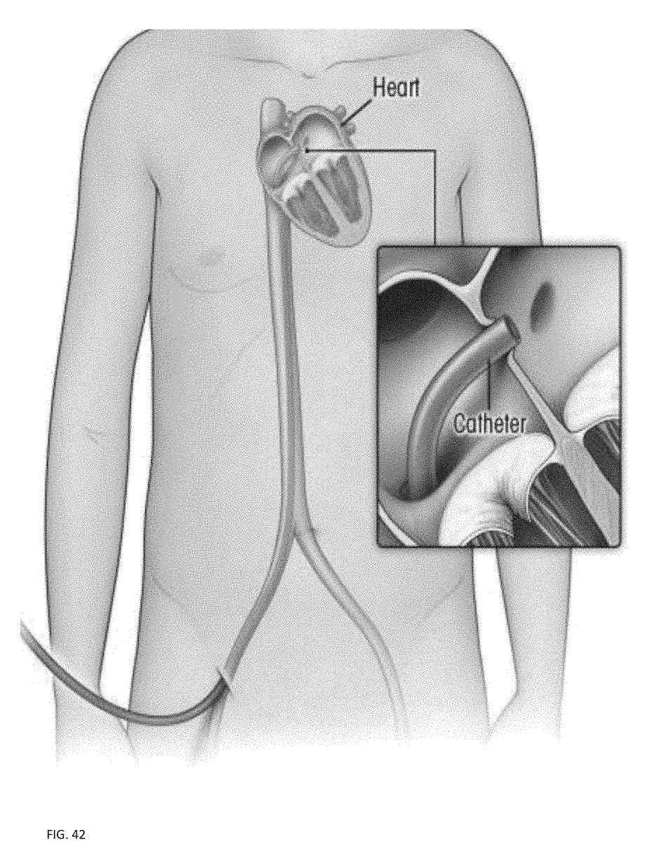

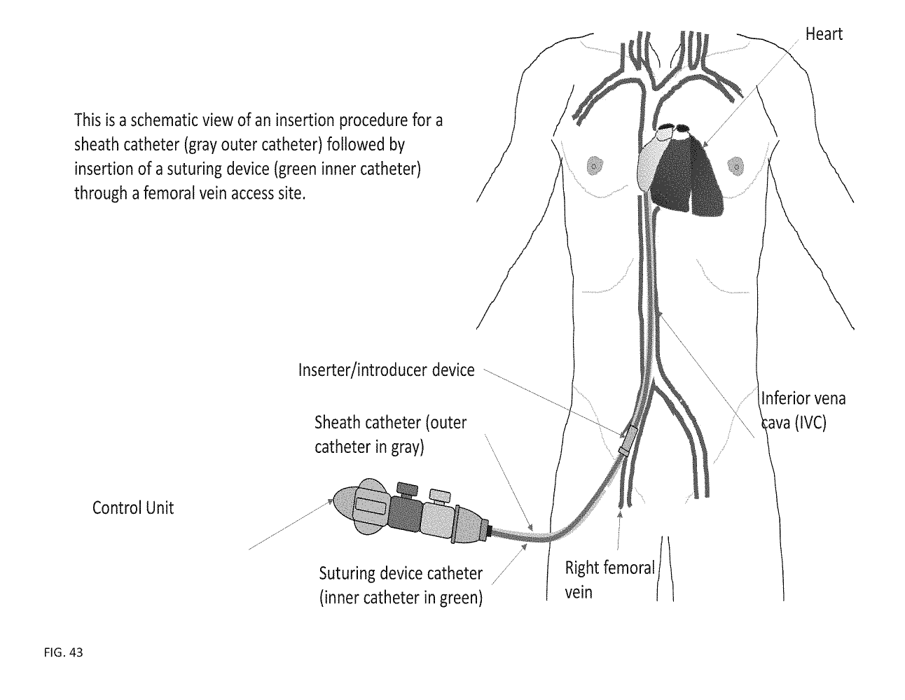

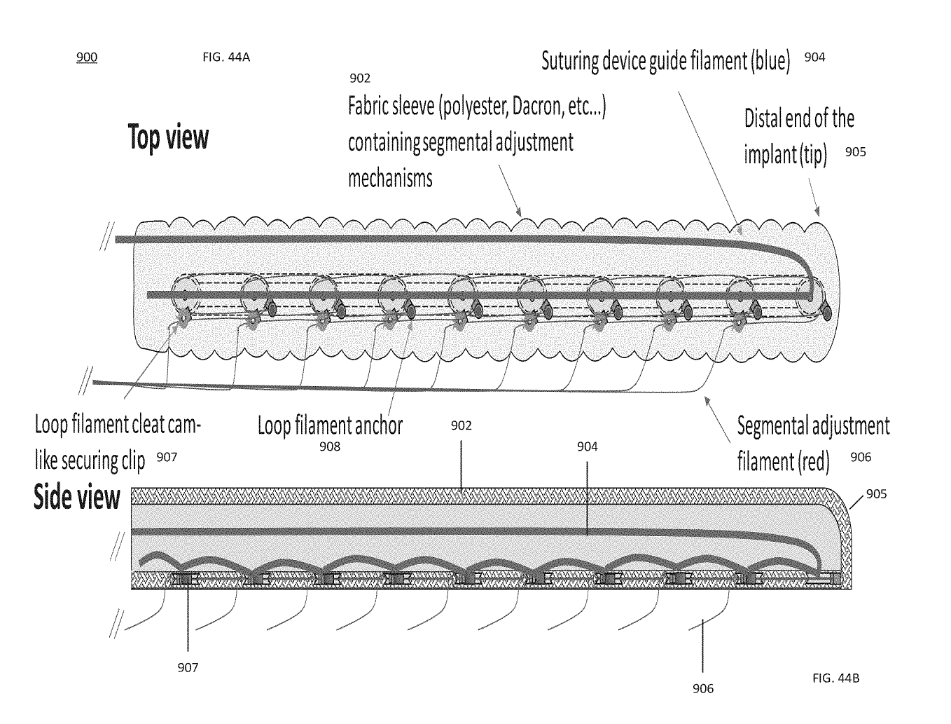

In some embodiments, based on FIG. 6A, the SATAR device is an initial flexible flat ring (e.g., the body 202) including a linear assembly of interlocking segments (e.g., the segments 204), each of which is comprised of a short central hollow tubular section connecting a proximal and a distal coil wound in opposite (inverted) directions relative to each other. The SATAR device can be similar to two corkscrews of opposing pitches placed at 180 degrees from each other and joined in that orientation at their bases by a short hollow tube. These proximal and distal coils are fixed to the central section, meaning that they will not move. As shown in FIGS. 28-29, and 32-36, a moveable coil having the same diameter and winding direction of its corresponding fixed coil is wound into its corresponding fixed coil on each side (proximal and distal sub-segments) of the segment, forming a pair of interwoven coils on each side. These coil pairs are mated together by winding one coil into the other when the moveable coil is turned. The pitch of the coil pairs is inverted on the distal sub-segment of each segment compared to the proximal sub-segment. The distal moveable coil of one segment connects to the proximal moveable coil of the next segment by a flexible sleeve having freely rotating junctions connecting it to both coils. There is a suture fixation point (e.g., the eyelet 208) at the center of each segment (fixed to the central section) for suture fixation to the annulus 216. A mechanical principle applied in the SATAR device is similar as in a turnbuckle, as shown in FIG. 30 and FIGS. 31A-31B, where there is a central threaded sleeve mated to two screws, one coming in from each end of the sleeve. One screw has a right-hand thread and the other one a left-hand thread. Turning the sleeve in one direction will draw both screws closer together further into the sleeve, shortening the overall length of the turnbuckle. Turning the sleeve in the opposite direction will draw both screws out and farther apart from each other, lengthening the overall length of the turnbuckle. Transcatheter delivery of the SATAR device may require a small or smallest possible diameter. Therefore, the same turnbuckle principle is applied to the SATAR device design by changing each of the threaded portions of the sleeve and its corresponding screw for coils of the same size that would thread into the empty spaces of each other. The two sides of this “turnbuckle” would join at the center (such as two corkscrews facing in opposite directions joined together at their bases). A technically beneficial feature of the SATAR device is that the SATAR device can be delivered as a linear structure that can be inserted through a catheter. The suture fixation point (e.g., the eyelet 208) would extend from the central section of each segment (e.g., the segments 204). This feature allows for suture fixation of each segment of the SATAR device to a different portion of the annular circumference, creating a ring-type structure with the desired anchor points against which segmental contraction of the SATAR device can effect a tailored contraction of different portions of the annulus 216. Winding of a moveable coil into or out of its corresponding fixed coil can be achieved through the engagement of inner ridges projecting towards the lumen of the moveable coil by a flexible shaft traversing the lumen of that moveable coil. Because the SATAR device is comprised of coil assemblies and hollow tubular sections, a lumen is formed. A flexible drive-shaft with a flattened spatula-like distal end is fed through the lumen of the SATAR device and aligned with the inner ridges of the proximal or distal movable coil within each segment. Rotating the flexible drive-shaft in one direction would engage the inner ridges of the moveable coil and rotate that coil accordingly. If, for example, clockwise drive-shaft rotation makes the moveable coil wind itself further into its corresponding fixed coil then the overall length of that coil pair, and therefore that proximal or distal sub-segment, would shorten. It follows that counterclockwise rotation would have the opposite effect, lengthening the overall sub-segment length. To minimize at least some unintended migration of the flexible drive-shaft during SATAR device deployment a holding chamber has been included at the distal end of the SATAR device. This holding chamber is designed to secure the spatula-like distal end of the flexible drive-shaft during deployment. An intentional simultaneous rotation and pulling of the flexible drive-shaft would be desired to align the spatula-like distal end of the shaft to the opening of the holding chamber in order to release it into the lumen of the SATAR device. The SATAR device segments and sub-segments would then be shortened or lengthened as needed from distal to proximal but the flexible drive-shaft can move back and forth within the lumen, as needed, to re-engage any sub-segment, as needed. Because the SATAR device is comprised of multiple segments, and each segment has proximal and distal sub-segments, significant flexibility is built into the SATAR device to allow for tailored segmental (and sub-segmental) annular lengthening or shortening. Such versatility maximizes a capability of a surgeon to optimize the SATAR device geometry according to individual patient anatomy in order to achieve the desired reshaping of the annulus 216 of the mitral valve. Another potential benefit of the SATAR device is that it is comprised of linear segments that could be made from material of variable stiffness so as when contracted the stiffness is increased. The SATAR device enables fine-tuning of the ring size and shape after implantation which allows the surgeon to optimize the size and the coaptation length with real-time feedback to improve surgical outcomes. The SATAR device is similar in size and shape to standard commercially available rings, but can be implanted by a transcatheter approach.

In some embodiments, based on FIGS. 1-6B, the SATAR device can be transcatheter implanted as follows. The inguinal area (presumably on the right side) is identified and thoroughly cleaned. A surgical field is established. After the surgical field has been prepped and draped as usual, the inguinal area is palpated and the femoral vein is punctured using either anatomical landmarks, ultrasound guidance, or other means of identification. A guide wire is inserted through the access needle, the needle is removed and the Brockenbrough (or equivalent) needle is inserted. After positioning at the superior vena cava, the first movement seen after pulling back the Brockemrough needle is the catheter falling into the right atrium from the SVC and followed by a second more subtle movement as the catheter falls from the thicker muscular intra-atrial septum into the fossa ovalis. The assembly (e.g., needle plus guidewire) is then gently advanced and, if its the position is correct, then the assembly may catch on the lip of the fossa. At this point, the pressure tracing should demonstrate a higher pressure with a straight line suggesting the tip of the catheter is abutting the intra-atrial septum, thus damping the pressure tracing. After verifying the right position, the septal puncture should be performed and the needle advanced into the transseptal sheath. Once satisfied that the needle is properly positioned, a dilator is passed over the wire in preparation for subsequent steps. At this time, an introducer is inserted through the venous access port. A steerable sheath catheter containing a tapered tip stylet is inserted through the introducer and navigated under fluoroscopic guidance up the left atrium. From this step onward, the remainder of the procedure will be carried out under 3D echocardiography guidance. The suturing device, in its closed (hinged) configuration is loaded with the curved needle and trailing suture thread, leaving only the tip of the needle exposed. The SATAR device is loaded into the suturing device by fitting the suturing device needle tip (already exposed) through the SATAR device's most distal suture anchor slot (anchoring orifice). The loaded suturing device and attached SATAR device are both introduced through the steerable sheath catheter and advanced out of the sheath into the left atrium. Under 3D echo guidance the mitral valve is identified. The anterolateral trigone and anterolateral extreme of the posterior leaflet's P1 segment are identified along with the corresponding segment of the annulus. The suturing device, loaded with suture and the SATAR device, is positioned facing the corresponding segment of the annulus. At this time the suturing system is fully deployed (hinge fully opened). The fully deployed suturing device is apposed against the annulus and the device actuated to drive the suture needle and thread through the annulus. The needle is recaptured into the suturing device and withdrawn from the field along with the suturing device. The SATAR device remains apposed to the annulus, but still not fixed to the annulus. Extracorporeally, both ends of the suture thread (identified by different colors for each half of the suture thread) are tied together to form one loop (throw) of the surgical knot. This loop is loaded into spiral knot pusher. The process is repeated with subsequent loops being loaded more proximally along the spiral knot pusher until the desired number of throws is loaded. The loops may be loaded from opposing sides to minimize the occurrence of the loops being disengaged from the knot pusher when exerting tension or going around a curve. Please note that a cutting ring (with internal pegs engaging it to the spiral knot pusher) has already been preloaded on the spiral knot pusher proximal to the last suture loop. The loaded knot pusher is advanced through the introducer, sheath catheter and into the left atrium while maintaining tension on the suture threads. The thread loops will slide along the length of the suture thread as they are pushed by the knot pusher. Once the knot pusher reaches the pierced annular segment, the knot will be turned (rotated) on its axis as to move the knot throws (loops) distally until each loop is delivered under tension to the tissue, forming and tightening the knot. Continued rotation of the knot pusher will drive the cutting ring distally until its cutting edge is forced against the suture threads until they are cut. At this point, the first anchoring knot has been delivered and the distal end of the SATAR device is fixed to the annulus. A new suture needle and thread are loaded into the closed (hinged) suturing device as detailed above and the suturing process repeated at each suture anchoring point of the SATAR device. Once the last anchoring suture has been delivered, the suture device (e.g., the threading device 100) is withdrawn and the device is ready to effect segmental contraction of the annulus. Under 3D echo guidance, the flat head of flexible shaft (residing in the terminal reservoir during implantation) is moved proximally until the flat head engages the inner ridges of the moveable coil belonging to the most distal adjustable segment. Note that the flexible shaft is marked at its proximal end to allow for its precise displacement and coupling with the inner ridges at different segments. Similarly, the SATAR device segments are designed to be identified using 3D echo or fluoroscopy. The flexible shaft is turned, turning the coil and contracting the most distal segment of the SATAR device. This action in turn contracts the corresponding segment of the annulus to which the SATAR is fixed. Once the most distal segment has been adjusted then the flexible shaft is pulled more proximally until the next segment is engaged and contracted as desired. The process is repeated under 3D echo visualization until the desired amount of contraction (or expansion if desired) is attained and a tailored, segmentally optimized annuloplasty has been achieved as per real-time 3D echo parameters. The entire process is carried out with a beating heart and with the possibility of performing immediate adjustments echo-guided to maximize the efficiency and therapeutic benefit of the procedure.

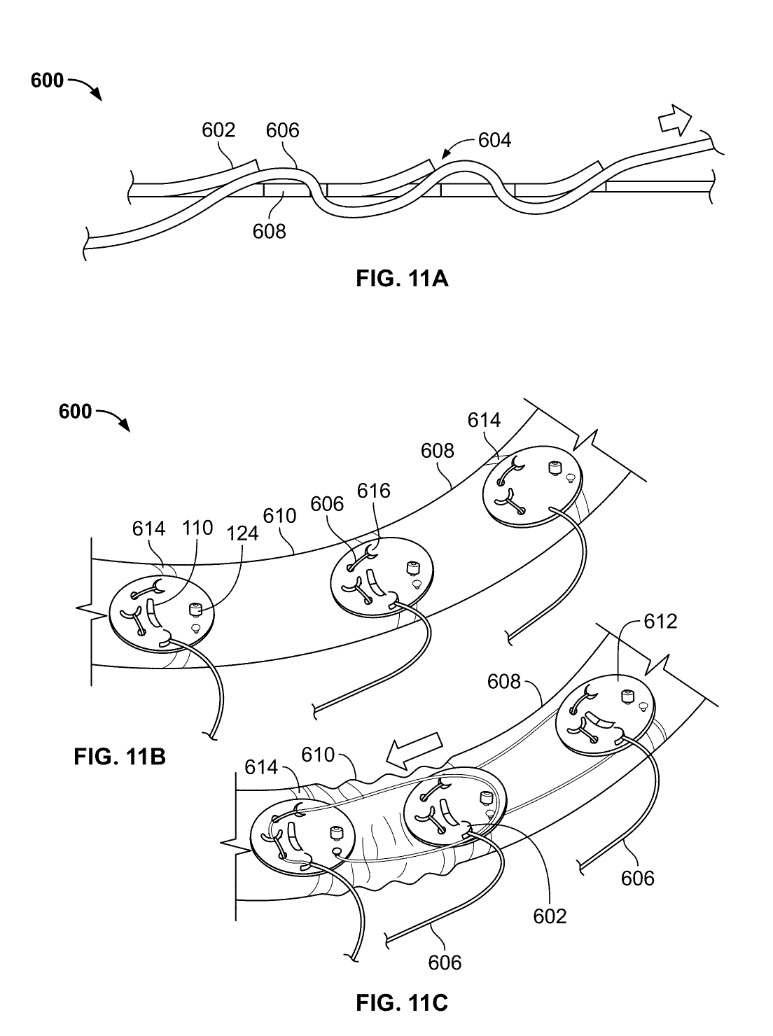

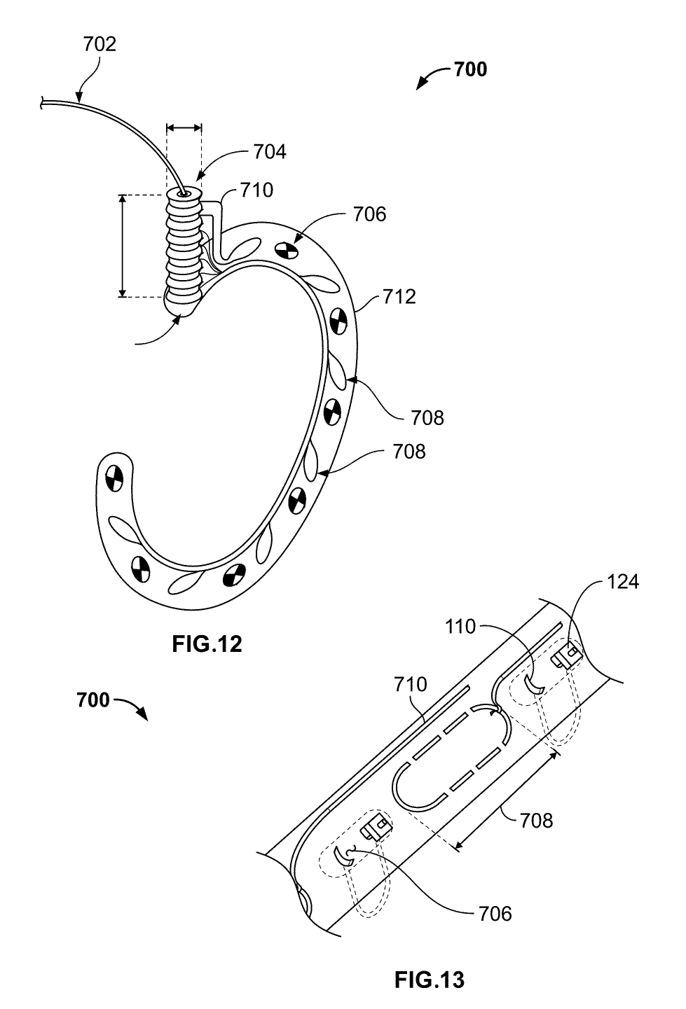

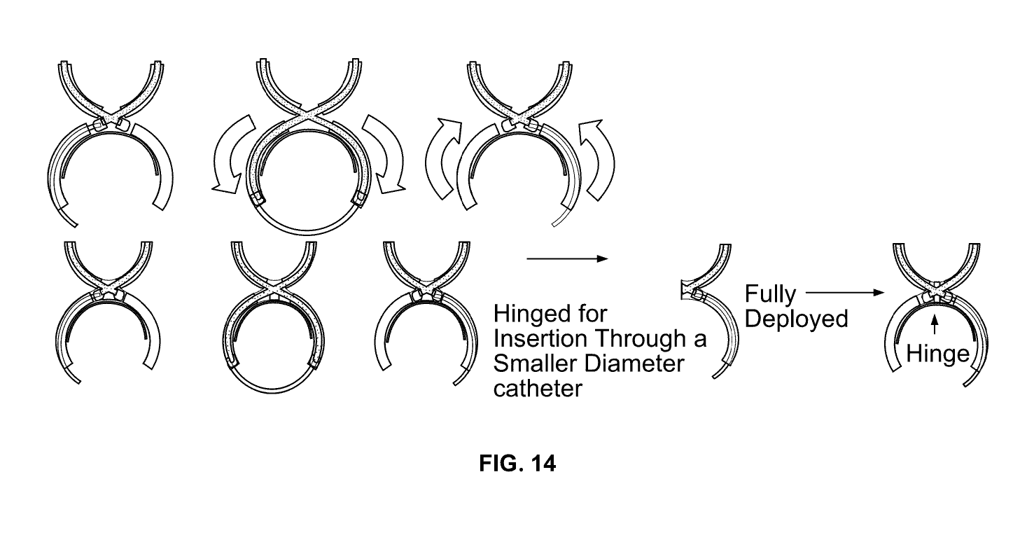

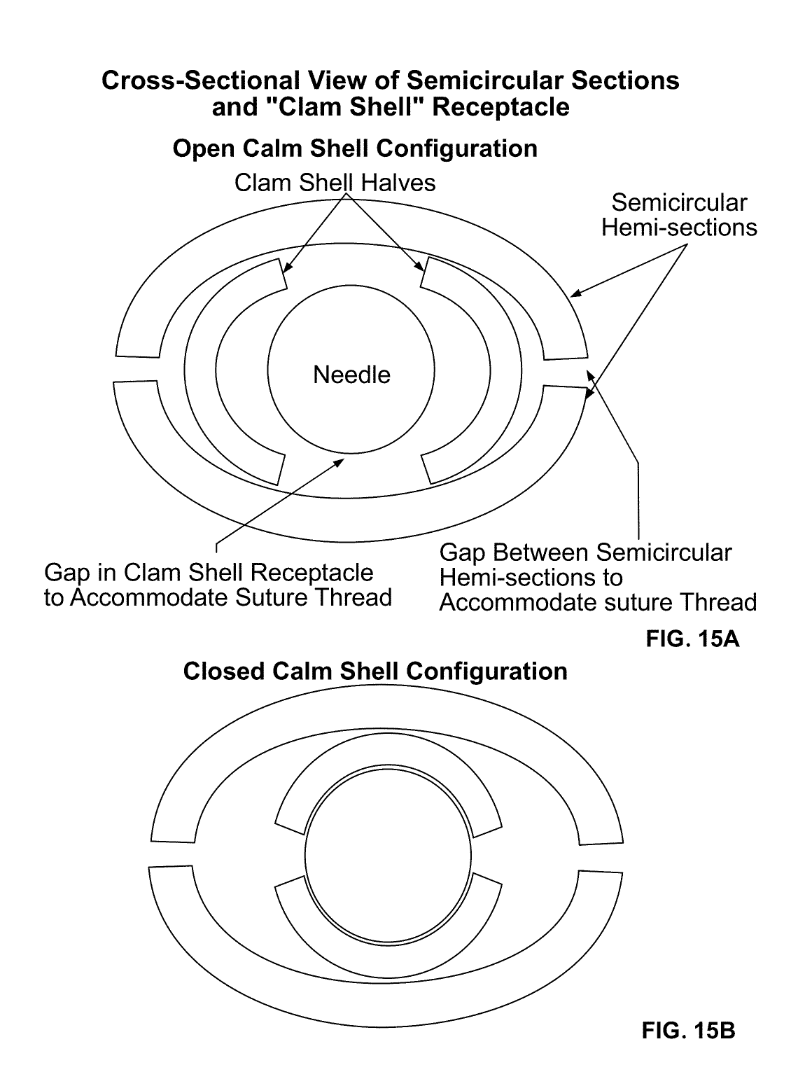

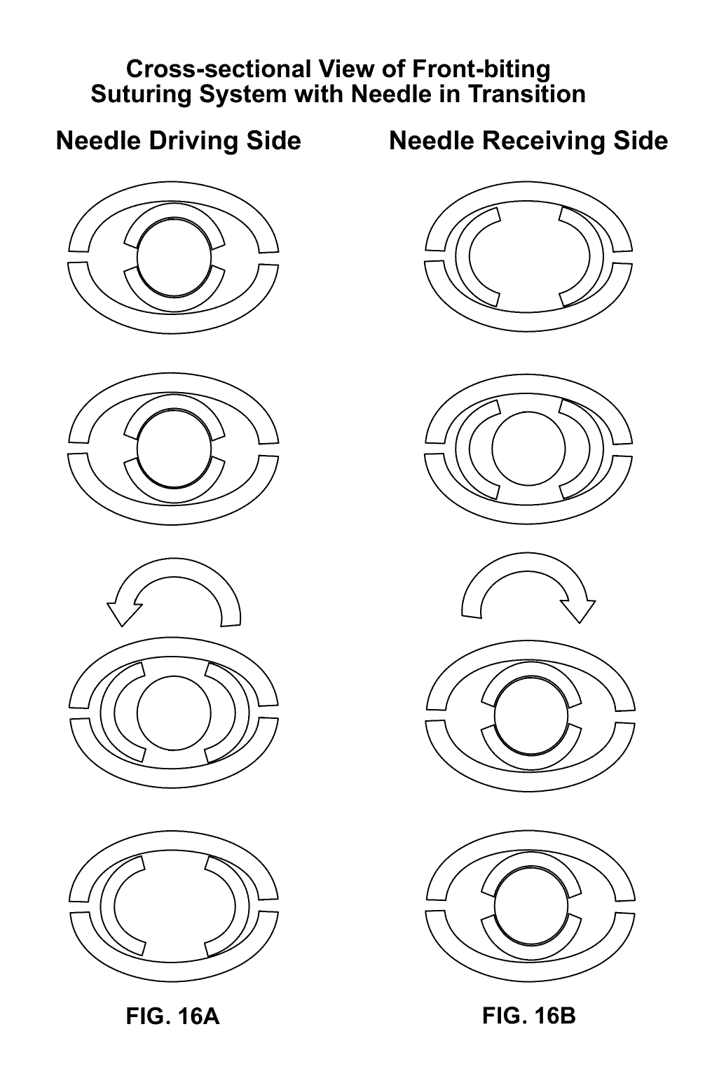

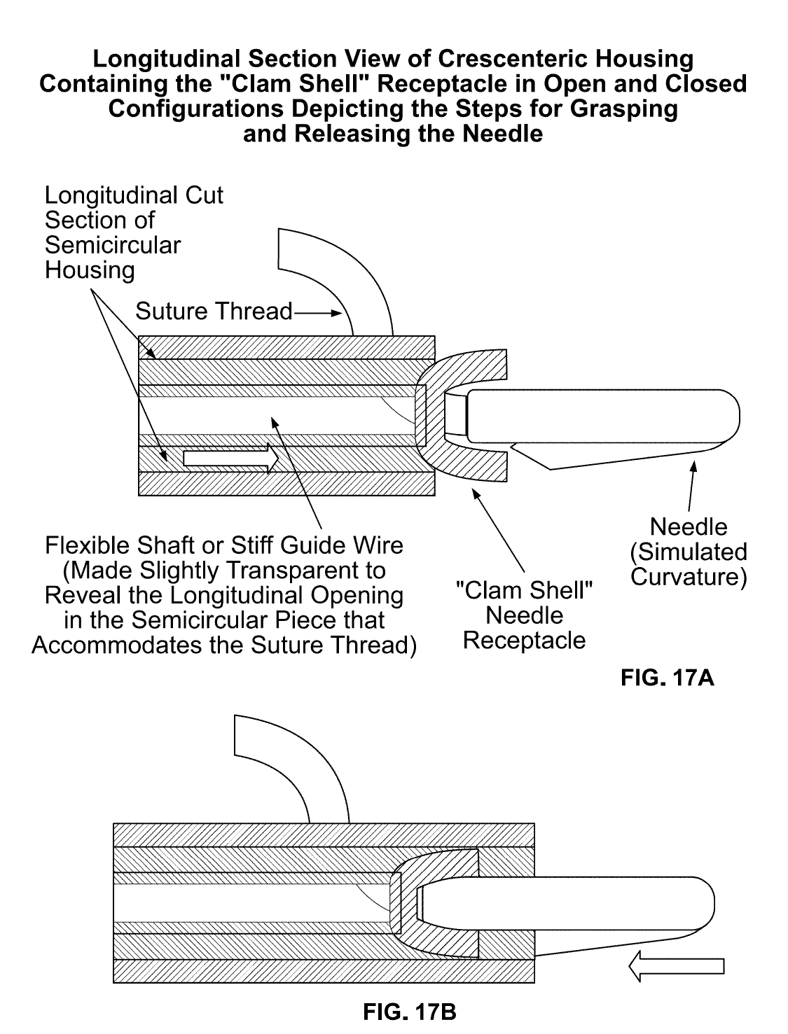

FIGS. 8A-8C show a plurality of perspective diagrams of a technique for adjusting an elongated member in accordance with this disclosure. FIGS. 9A-9D show a plurality of perspective diagrams of a technique for adjusting an elongated member in accordance with this disclosure. FIGS. 10A-10C show a plurality of perspective diagrams of a technique for adjusting an elongated member in accordance with this disclosure. FIGS. 11A-11C show a plurality of perspective diagrams of a technique for adjusting an elongated member in accordance with this disclosure. FIGS. 12-13 show a plurality of perspective diagrams of a technique for adjusting an elongated member in accordance with this disclosure. In particular, these various embodiments all share a common characteristic of being able to change rigidity based on various techniques including but not limited to mechanical techniques, heating techniques, bending techniques, via intrinsic properties of an elongated member material, or others. For example, with regard to some of the intrinsic properties of the elongated member material, such embodiments can include Nitinol or other types of alloys that exhibit shape-memory effect or super elasticity or other non-metal and non-alloy based materials. For example, some tensioning can be controlled via pull strings manipulated at the proximal end of catheter or with pull strings located permanently in-situ, but manipulated remotely. For example, a SATAR type design allows an adjustment of an annuloplasty ring configuration to optimize mitral valve leaflet coaptation to restore a physio-anatomic D-shape of an annulus of the mitral valve, expect same good results as surgical implanted rings and better clinical outcome in patients with functional mitral regurgitation. For example, because of the nature of its design, a SATAR type device can also be used for correction of a tricuspid valve regurgitation as (1) more than 20% of patients undergoing surgery for mitral valve disease have significant functional tricuspid regurgitation and (2) annular encircling and bands have proven to be less effective as tricuspid annuloplasty rings.

As shown in FIGS. 8A-8C, an elongated member 300 (e.g., tubular sleeve, tubular sock) is output (e.g., introduced, telescoped) from a tube 302 (e.g., catheter). The elongated member 300 includes a distal end portion 304, which can be open or closed. The elongated member 300 includes a plurality of segments 306 that are interconnected (e.g., interlinked, fastened, mated, interlocked, bracketed, magnetized, adhered) with each other such that the segments 306 are selectively, individually, and independently adjustable in position, orientation, angling, diameter, or other geometric characteristics. The segments 306 are segmented via a plurality of borders 308, which can be external or internal to the elongated member 300. The elongated member 300 hosts a plurality of islands 316, which can extend across at least two of the segments 306 at the border 308. The islands 316 are unitary to or assembled with the elongated member 300. At least some of the islands 316 can host the toggle 110 and the lock 124. The islands 316 host a plurality tension sutures 314 having a plurality of tails 310. The tension suture 314 are adjustable via a plurality of clips 312, which are U-shaped, C-shaped, horseshoe-shaped, crescent-shaped, or others. At least some of the clips 312 are tension clips and can have a pair of legs, one of which can be movable relative to the other in order to adjust the tension sutures 314.

In some embodiments, based on FIGS. 1-7B, the elongated member 300 can be used with three catheters that work together: a guide catheter, a suturing/implant delivery catheter, and an adjustment cutting catheter. The elongated member 300 can be a woven Dacron like tube having an interior (e.g., cavity, layers, pockets) with some regions on the interior that are modified with areas that are bonded to form the islands 316 as well as having the clips 312 that are integrated. The elongated member 300 (e.g., implant) is sutured down incrementally (e.g., sock stays at distal end while suture catheter removed and reloaded). The clips 312 can be temporarily or permanently attached to the elongated member 300 (e.g., implant) and have a low profile. The clips 312 can reside between the borders 308 or the islands 316 (e.g., suture device can slide by clips 312 in guide catheter based on geometry). The toggles 110 are muzzle loaded in the suturing catheter. The elongated member 300 exhibits puppeteer style of control, effected from a proximal end of the catheter (e.g., via surgeon). For example, individual sutures are tensioned by pulling outside of patient or with adjustment catheter. Loosening is accomplished by introducing a catheter over suture that levers open clip to allow suture to go slack. Guide wire type construct on the proximal end of the suture to allow ease of use. Cut off catheter used to cut each suture off after proper tension achieved. Free ends (e.g., tails 312) of tension sutures 314 can extend tangential or perpendicular to the elongated member 300 (e.g., implant). At least some of the clips 312 are toothed and can be rotated about 90° clockwise (or vice versa) and the free ends can extend to proximal end of the catheter for tensioning. One end of suture is can be coupled or affixed to the leg of clip 312. Alternatively, one or more of the clips can be rotated about 10 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, or 180 degrees, clockwise or counter-clockwise.

As shown in FIGS. 9A and 9D, an elongated member 400 (e.g., tubular sleeve, tubular sock) is output (e.g., introduced, telescoped) from a tube (e.g., catheter). The elongated member 400 includes a body 402 having a plurality of segments 404 that are interconnected (e.g., interlinked, fastened, mated, interlocked, bracketed, magnetized, adhered) with each other such that the segments 404 are selectively, individually, and independently adjustable in position, orientation, angling, diameter, or other geometric characteristics. The segments 404 are segmented via a plurality of borders 414, which can be external or internal to the elongated member 400. The elongated member 400 hosts a plurality of anchors 406 positioned over the borders 414 between the toggles 110 and the locks 124, although at least some of the anchors 406 can be positioned between the borders 414. The anchors 406 host a plurality of channels 412 that are tapered or notched (e.g., V-shaped) longitudinally tapered relative to the elongated member 400. The anchors 414 host a plurality of cords 408, where each of the anchors 414 hosts at least two of the cords 408 such that these at least two cords 408 are positioned or offset next to each other and at least one of the at least two cords 408 is hosted via channel 412 of that respective anchor 406. The cords 408 can be compressible, elastic, resilient, or others. The cords 408 can include plastic, fabric, rubber, silicon, or other suitable materials. Each of the cords 408 has an end portion 410 (e.g., spherical, ovoid, cuboid, beaded, knotted) that is larger or more volumetric than the channels 412 such that the end portion 410 prevents the cords 408 from sliding through the channels 412 (and the anchors 406). For example, the cords 408 can be pulled relative to the anchors 408 and wedged laterally relative to the anchors 408 (e.g., pull into the channels 412 to secure).

In some embodiments, based on FIGS. 1-7B, the elongated member 400 (e.g., sock) can be used with three catheters that work together: a guide catheter, a suturing/implant delivery catheter, and an adjustment cutting catheter. The elongated member 400 (e.g., implant) is a woven Dacron like tube and the anchors 406 (e.g., cleats) are designed to be attached to the elongated member 400, but allow a suturing head to pass by while in the guide catheter. Location of the suture would be informed by the position of the anchor 406 (e.g., cleat) when the suture is near the anchor 406. The elongated member 400 can be sutured down incrementally (e.g., sock stays at distal end while suture catheter removed and reloaded). The anchors 406 (e.g., cleats) can be temporarily or permanently attached to elongated member 400 and have a low profile. The anchors 406 may have a sub structure at each node, bands or molded features that attach the anchors 406 cleat to the elongated member 400. The anchors 406 are attached to each other with segmental tightening sutures in series (e.g., suture device can slide by cleats in guide catheter based on geometry). The toggles 110 are muzzle loaded in the suturing catheter (e.g., the threading device 100). The elongated member 400 exhibits a puppeteer style of control, effected from the proximal end of the catheter (e.g., surgeon). The individual sutures are tensioned by pulling outside of patient or with adjustment catheter. Loosening is accomplished by introducing a catheter over suture that has a specialized tip or manner to interact with the anchor 406 to allow un-cleating and retightening. Compromise between scale of cleat and finesse needed in cleat and uncleating tool. Guide wire type construct on the proximal end of the suture to allow ease of use. Cut off catheter used to cut each suture off after proper tension achieved.

As shown in FIGS. 10A-10C, an elongated member 500 (e.g., tubular sleeve, tubular sock) is output (e.g., introduced, telescoped) from a tube (e.g., catheter). The elongated member 500 includes a body 502 having a plurality of segments 504 that are interconnected (e.g., interlinked, fastened, mated, interlocked, bracketed, magnetized, adhered) with each other such that the segments 504 are selectively, individually, and independently adjustable in position, orientation, angling, diameter, or other geometric characteristics. The segments 504 are segmented via a plurality of borders 516, which can be external or internal to the elongated member 500. The elongated member 500 hosts a plurality of anchors 506 (e.g., islands) positioned over the borders 516 between the toggles 110 and the locks 124, although at least some of the anchors 506 can be positioned between the borders 516. The anchors 506 host a plurality of J-shaped bars 508 having a plurality of teeth sets 510 (e.g., zip style tooth strip). The J-shaped bars 508 can include plastic, rubber, silicon, metal, or others. Each of the J-shaped bars 508 includes an elongated portion 516 and a base 512. The elongated portion 516 hosts a set of teeth 510. The base 512 defines a channel therein. The channel is configured to receive the elongated portion 516 such that a respective teeth set 510 of that respective elongated portion can enable that respective J-shaped bar 508 to operate as a ratchet (e.g., unidirectional, bidirectional) relative to another J-shaped bar 508. The base 512 includes an arm 514 (e.g., cantilevered arm) that is outwardly movable relative to the base 512 away from the elongated portion 516 such that the arm 514 enables the elongated portion 516 to be released (e.g., pull away and release) and thereby longitudinally movable back and forth, as needed. The bases 512 are coupled (e.g., mated, interlocked, fastened, adhered, knotted, stapled) to the anchors 506.

In some embodiments, based on FIGS. 1-7B, the elongated member 500 (e.g., sock) can be used with three catheters that work together: a guide catheter, a suturing/implant delivery catheter, and an adjustment cutting catheter. The elongated member 500 (e.g., implant) is a woven Dacron like tube having an interior where some regions on the interior are modified with areas that are bonded to form islands (e.g., the anchors 506) as well as having integrated zip tie like cleats. The elongated member 500 is sutured down incrementally (e.g., sock stays at distal end while suture catheter removed and reloaded and reintroduced). The clips (e.g., the anchors 506) are temporarily permanently attached to the elongated member 500 and have low profile. The clips reside between the segments 504 of the elongated member 500 (e.g., suture device can slide by clips in guide catheter based on geometry.) The toggles 110 are muzzle loaded in the suturing catheter (e.g., the threading device 100). The toggles 110 can be attached to base of clip or straddle node. Possible for clip to work with suture catheter to help locate clips. The elongated member 500 exhibits a puppeteer style of control, effected from the proximal end of the catheter. The individual sutures are attached to the zip tie ends and are tensioned by pulling outside of patient or with adjustment catheter. Loosening is accomplished by introducing a catheter over suture that levers open clip to allow zip tie ends to go slack. Guide wire type construct on the proximal end of the suture to allow ease of use. Cut off catheter used to cut each suture off after proper tension achieved.

As shown in FIGS. 11A-11C, an elongated member 600 (e.g., tubular sleeve, tubular sock) is output (e.g., introduced, telescoped) from a tube (e.g., catheter). The elongated member 600 includes a body 608 having a plurality of segments 610 that are interconnected (e.g., interlinked, fastened, mated, interlocked, bracketed, magnetized, adhered) with each other such that the segments 610 are selectively, individually, and independently adjustable in position, orientation, angling, diameter, or other geometric characteristics. The segments 610 are segmented via a plurality of borders 614, which can be external or internal to the elongated member 600. The elongated member 600 hosts a plurality of islands 602, which can extend across at least two the segments 608 at the borders 614. The islands 602 can be unitary to or assembled with the elongated member 600. At least some of the islands 602 can host the toggle 110 and the lock 124. The islands 602 host a plurality of tension tabs 616 that enable a plurality of suture portions 606 of a plurality of tension sutures 612 to extend through the suture tabs 616. The tension sutures 612 loop about the islands 602 such that the tension sutures 612 extend in a serpentine path relative to the islands 602 such that the serpentine path enables the tension sutures 612 extend between the islands 602 via the tension tabs 616, whether below or above the islands 602 (e.g., serpentine path with multiple single direction clamps).

In some embodiments, based on FIGS. 1-7B, the elongated member 600 (e.g., sock) can be used with three catheters that work together: a guide catheter, a suturing/implant delivery catheter, and an adjustment cutting catheter. The elongated member 600 (e.g., implant) is a woven Dacron like tube having an interior where some regions on the interior are modified with molded areas that are bonded to form islands (e.g., the islands 602) that can act as pulleys and having a series of integrated tabs (e.g., the tension tabs 616) that act as clips to grip the suture portions 606. The tension sutures 612 are threaded thru the tension tabs 616 with free ends extending to proximal end of catheter for tensioning. Tensioning is possible due to the multiple single direction clamps (e.g., the islands 602) holding the tension sutures 612. The elongated member 600 can be sutured down incrementally (e.g., sock stays at distal end while suture catheter removed and reloaded). The tabs (e.g., the islands 606) can be temporarily or permanently attached to elongated member 600 and have a low profile. For example, the tabs reside at the segments 610. The suturing device can slide by clips in guide catheter based on geometry. The toggles 110 are muzzle loaded in the suturing catheter. The elongated sleeve 600 exhibits a puppeteer style of control, effected from the proximal end of the catheter (e.g., via surgeon). The individual sutures 612 are tensioned by pulling outside of patient or with adjustment catheter. May need catheter to push-pull on suture to avoid stiction like effects (e.g., may employ a mechanical advantage like a ratcheting handle to give incremental control over the tension). Loosening is accomplished by introducing a catheter over suture that levers open clip to allow suture to go slack. Guide wire type construct on the proximal end of the suture to allow ease of use. Cut off catheter used to cut each suture off after proper tension achieved.

As shown in FIGS. 12 and 13, an elongated member 700 (e.g., tubular sleeve, tubular sock) is output (e.g., introduced, telescoped) from a tube (e.g., catheter). The elongated member 700 includes a body 712 having a plurality of anchor zones 706, each having the toggle 110 and the lock 124 with the thread spanning therebetween. The body 712 includes a plurality of implant traction zones 708 such that at least one of the anchor zones 706 is positioned therebetween. Each of the implant traction zones 708 hosts a lasso-style filament 710 having a noose and coupled to the body 712. The lasso-style filament 710 are wound to a spool 704 of a winch mechanism coupled (e.g., anchored, fastened, sutured, mated, interlocked) to an anatomy portion (e.g., hard tissue, soft tissue) of a patient (e.g., heart). The winch mechanism hosts a guidewire 702 coupled (e.g., fastened, knotted) to the lasso-style filaments 710.

In some embodiments, based on FIGS. 1-7B, the elongated member 700 includes a woven member (e.g., body 712) that has a mesh and tension sutures (e.g., lasso-style filaments 710) woven into the mesh between anchoring zones (e.g., anchor zones 706). Ends of tensioning sutures (e.g., lasso-style filaments 710) are brought to the winch mechanism (e.g., spool 704) mounted at distal end of strip. Tensioning and de-tensioning can occur as winch has bi-directional self-locking and unlocking feature. Square drive cannulated screwdriver wire attached to last catheter. Detents on wheel faces used to keep wheels tight. Wheel and suture device go out together. The elongated member 700 (e.g., sock) can be used with three catheters that work together: a guide catheter, a suturing/implant delivery catheter, and an adjustment cutting catheter. The elongated member 700 is sutured down incrementally (e.g., sock stays at distal end while suture catheter removed and reloaded). The toggles 110 are muzzle loaded in the suturing catheter.

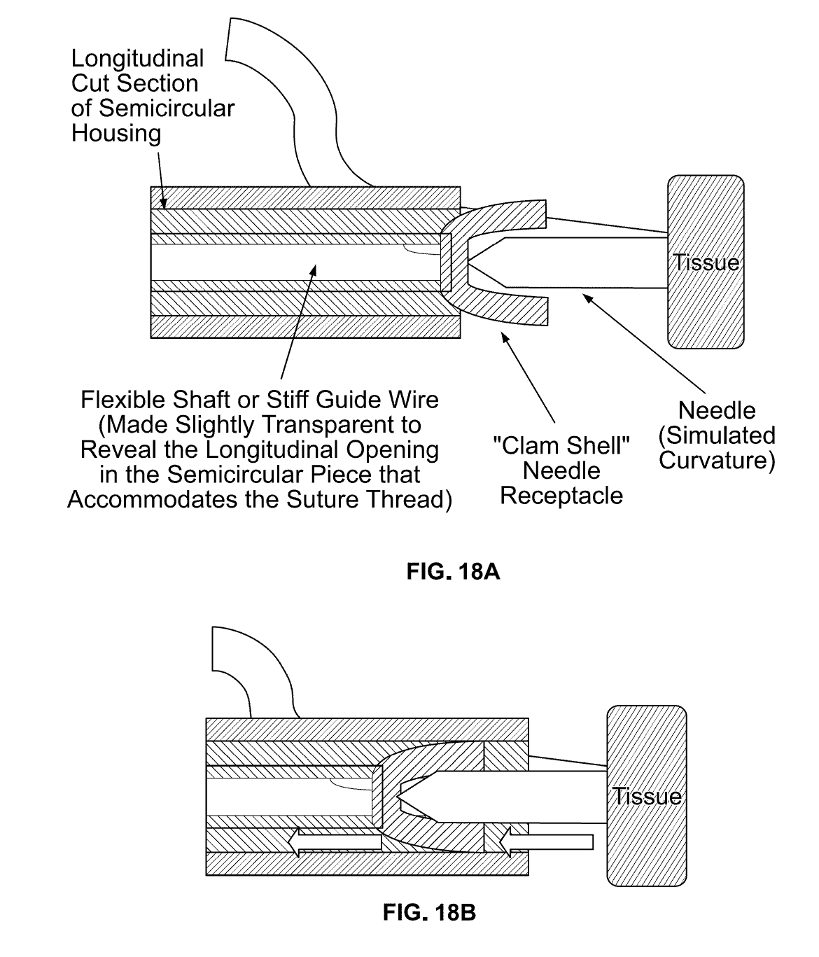

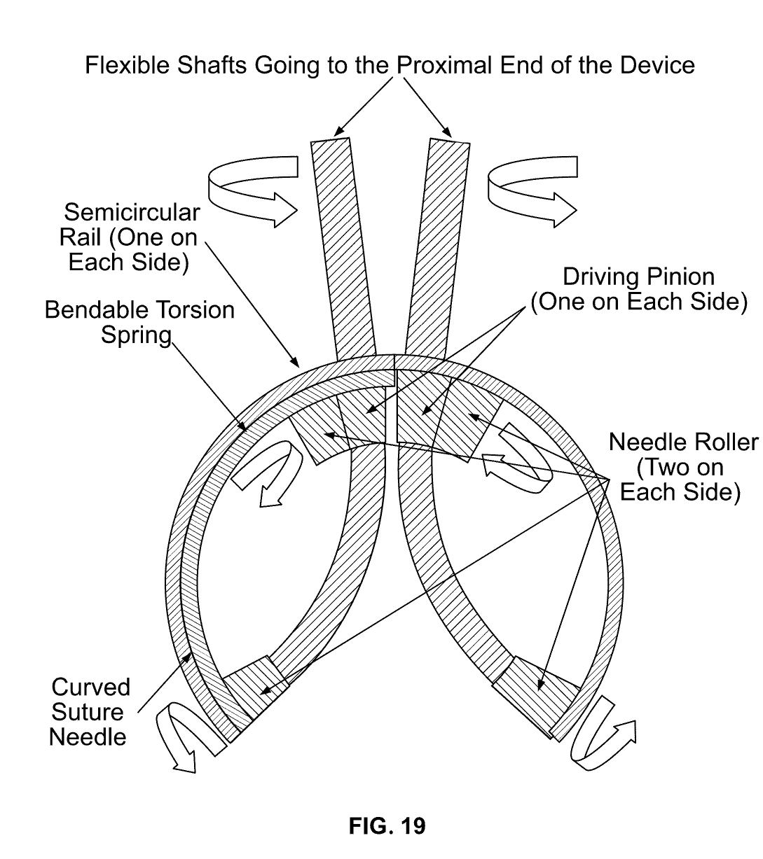

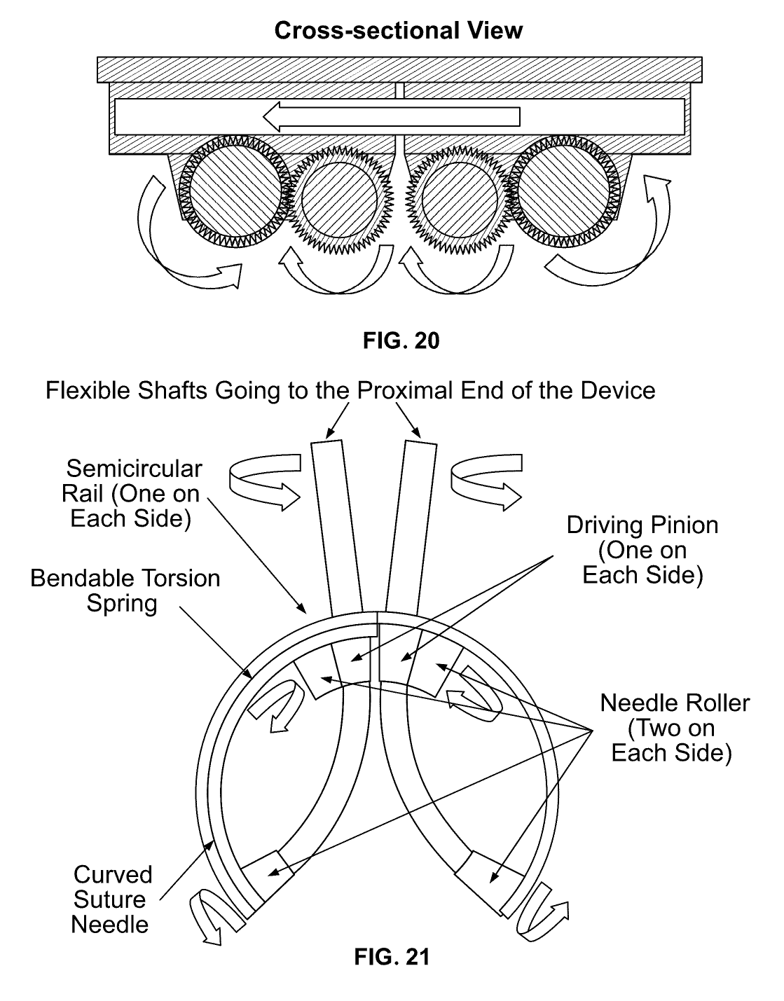

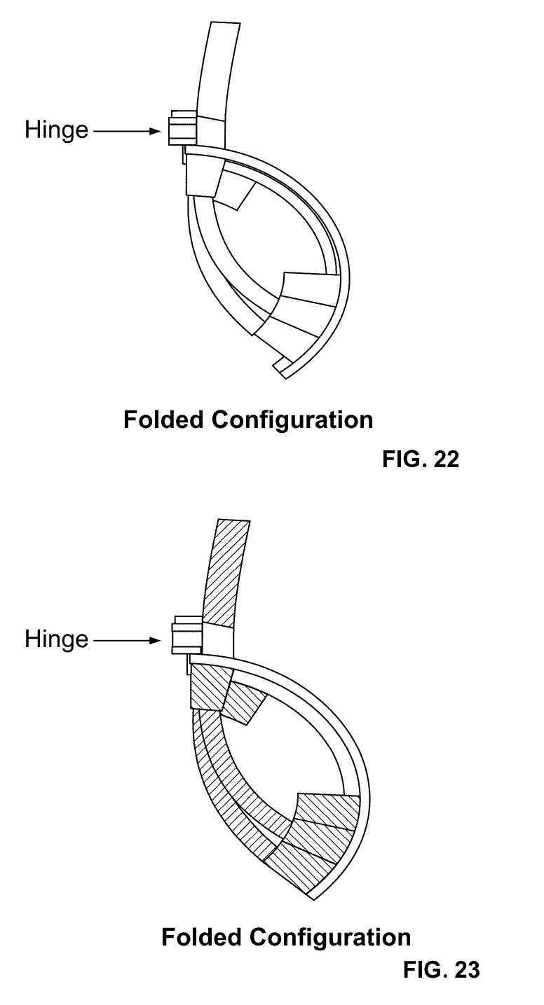

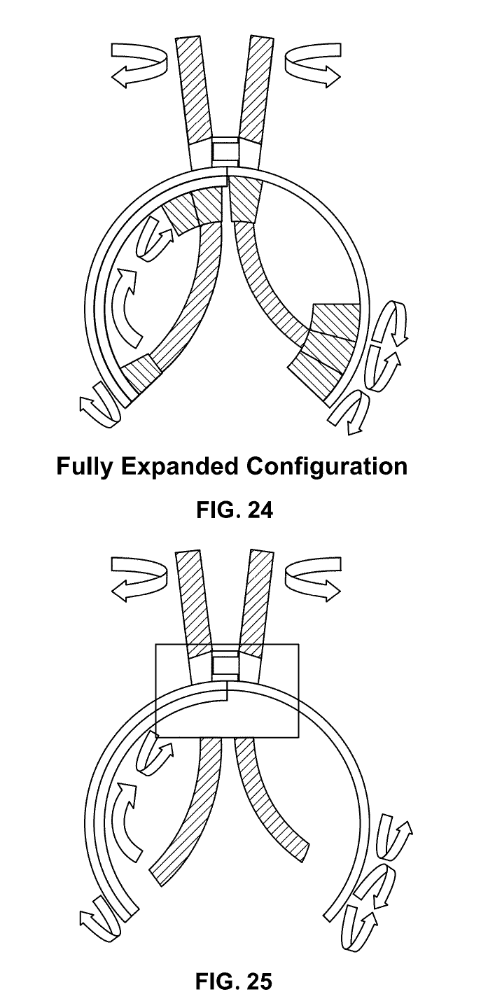

FIGS. 14-18B show a plurality of diagrams of a plurality of embodiments of front-biting suturing assembly for attaching an annuloplasty ring to a cardiac tissue in accordance with this disclosure. In particular, this suturing system 800 comprises flexible shafts or stiff guide wires coming from the proximal end (handle/control unit) of the device cross and end in opposite sides of a “horseshoe-type” needle housing structure. Each shaft ends in a clam shell-like needle receptacle that runs within the needle housing on each side, going back and forth from the proximal to the distal ends to push the needle out of the housing (through the tissue) or retrieve it and pull it in. Each “clam-shell” receptacle can be in either “open” or “closed configuration. In an open configuration the needle can slide out of the receptacle. In the closed configuration the receptacle would clamp the needle down to secure it. The design is meant to accommodate a curved suture needle within a horseshoe-shaped housing. This housing is comprised of two curved sections arranged like two incomplete semicircular sections, leaving a gap in the distal portion of the device wide enough to allow the needle to pass through the tissue, get captured and retracted back into the opposite side of the housing. Each section is itself partially divided longitudinally (as a croissant cut lengthwise but not completely divided) to have an anterior half and a posterior half). When you view the cross-section of the horseshoe and the receptacles they have an oval shape. The entire assembly is hinged lengthwise to minimize the diameter of the distal portion of the device and facilitate insertion through a smaller diameter catheter. One additional feature of this embodiment comprises a suturing system in which the entire distal end of the device or portions thereof are capable of rotation and needle positioning in any spatial direction relative to the rest of the device. The entire distal end of the device or portions thereof are also capable of pivoting the needle and/or needle housing structures so as to allow these to point the needle in any given direction or spatial plane without requiring rotation of other portions of the device,

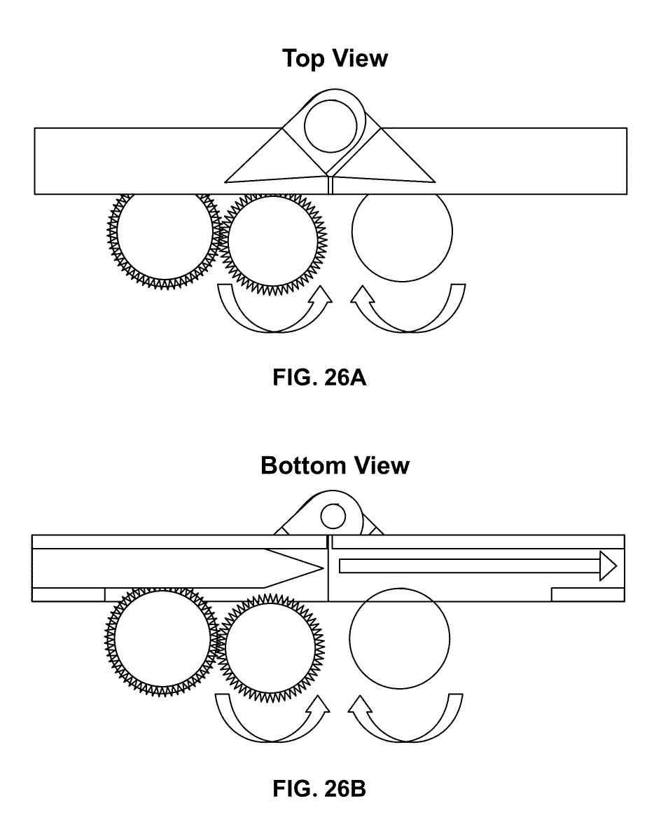

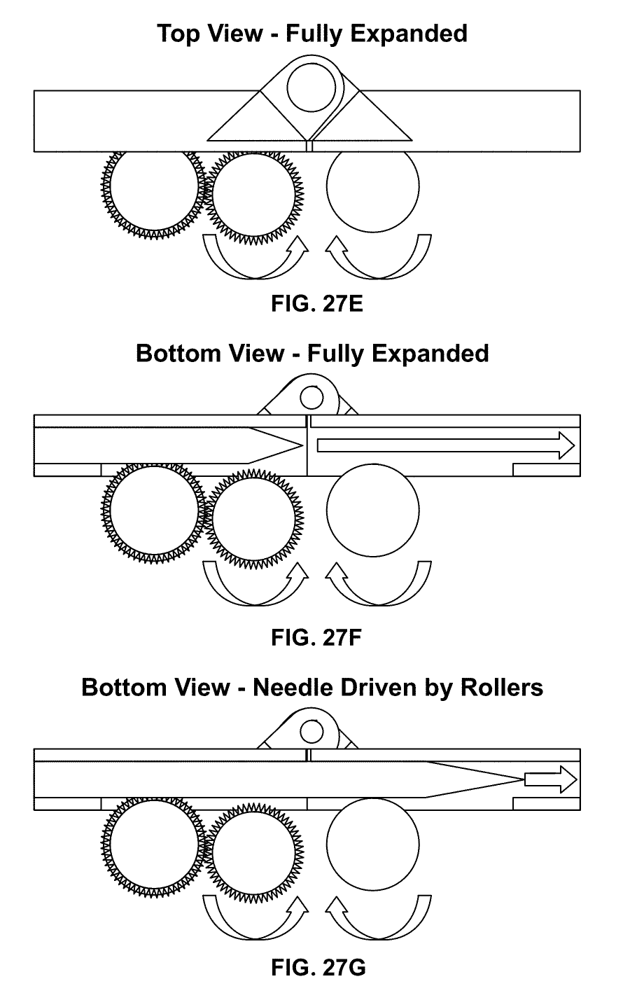

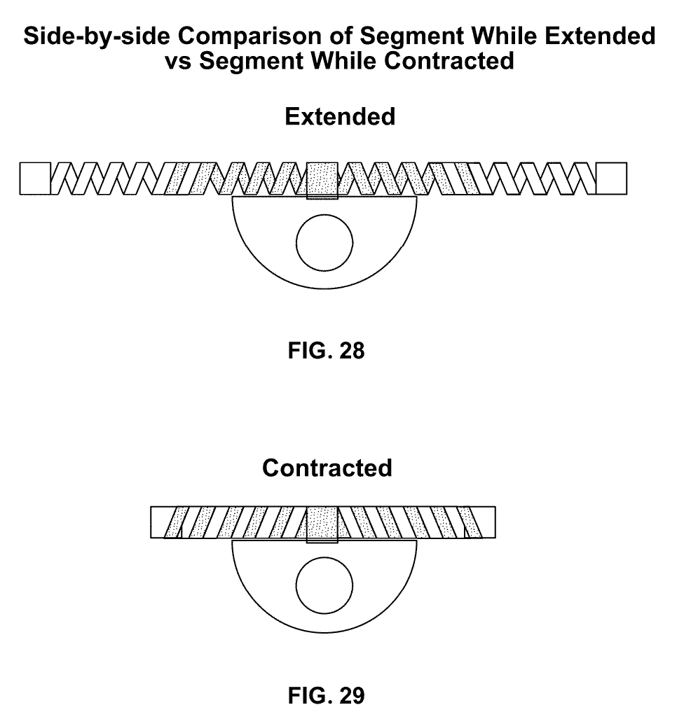

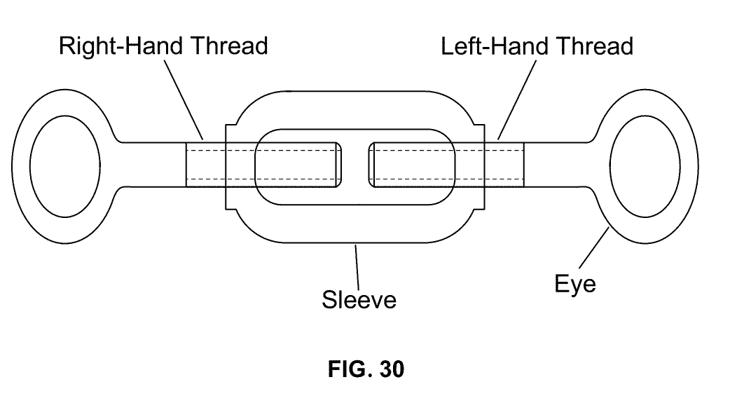

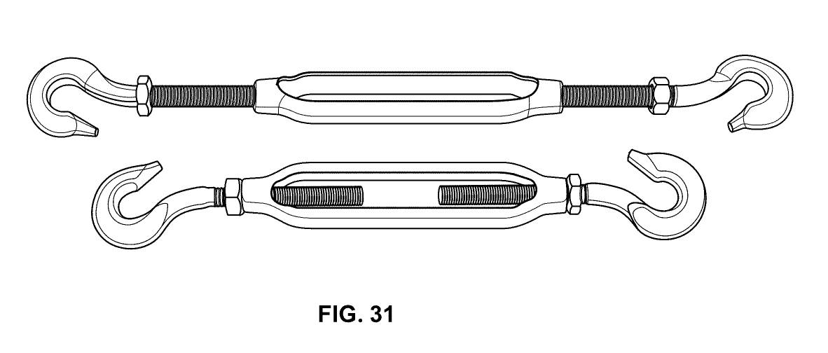

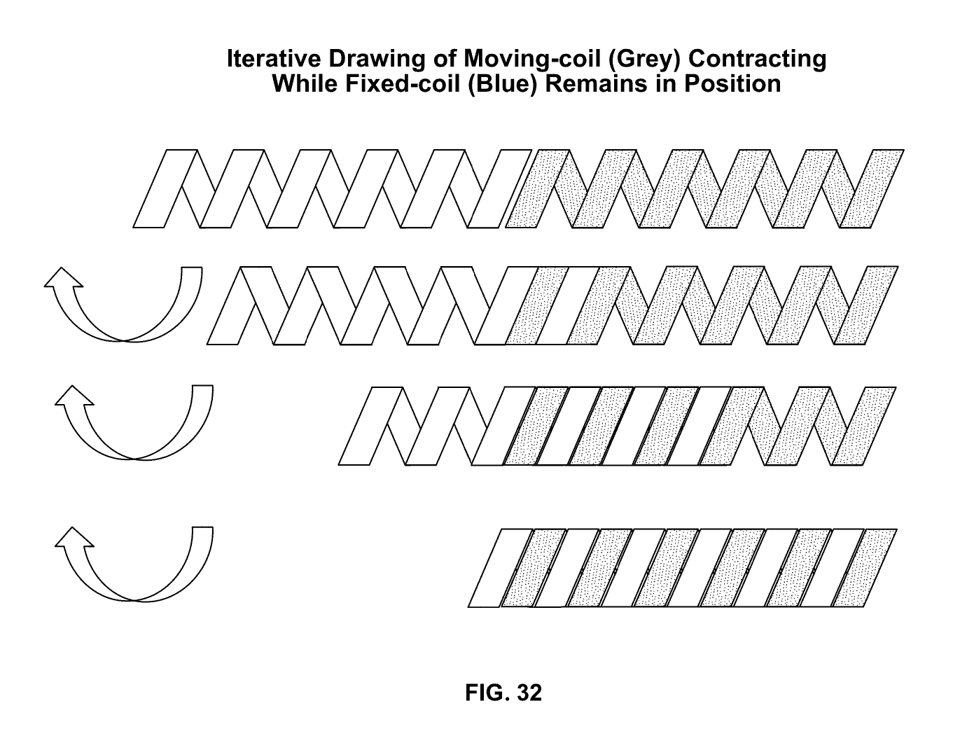

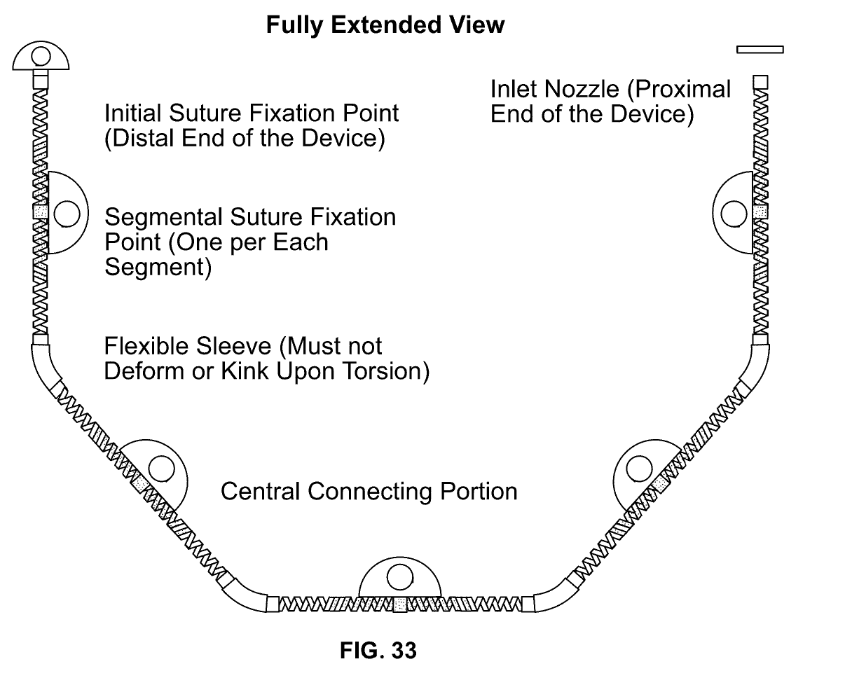

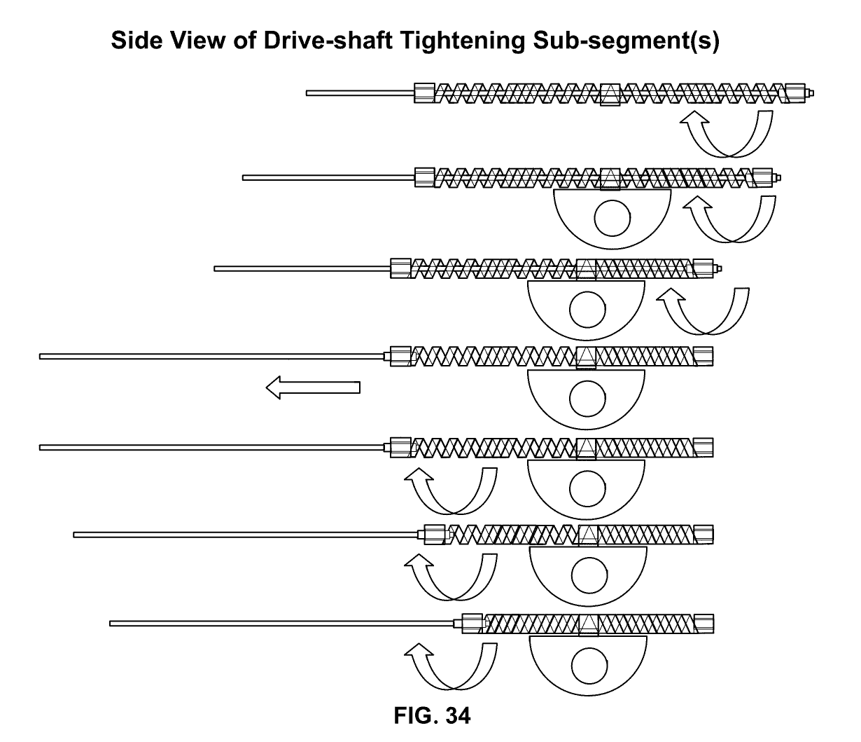

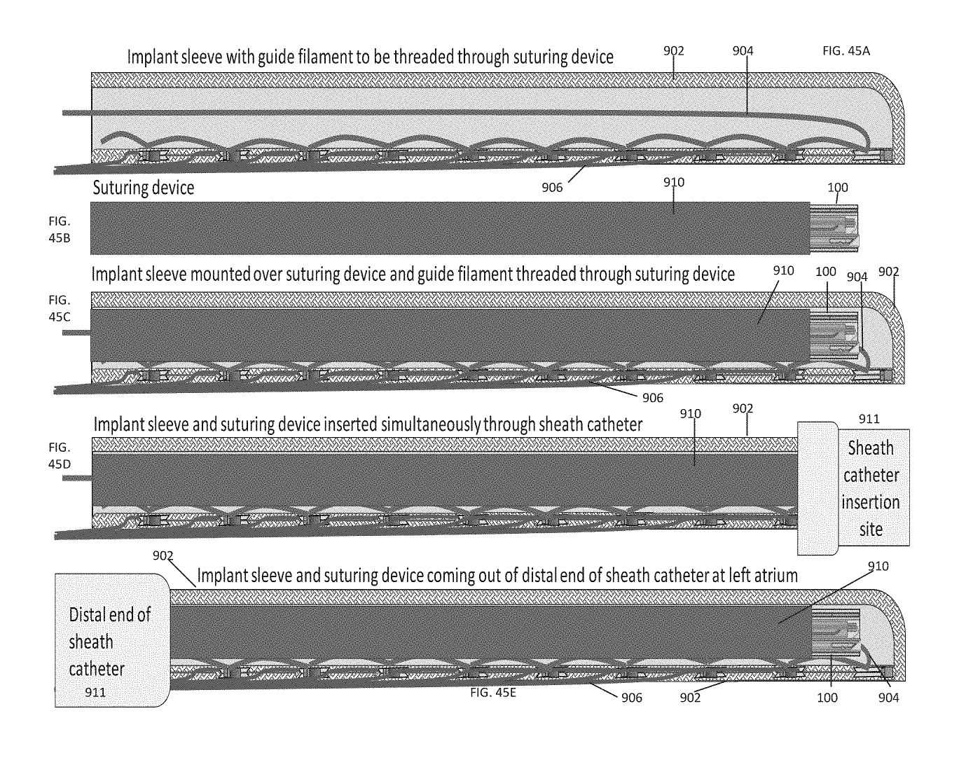

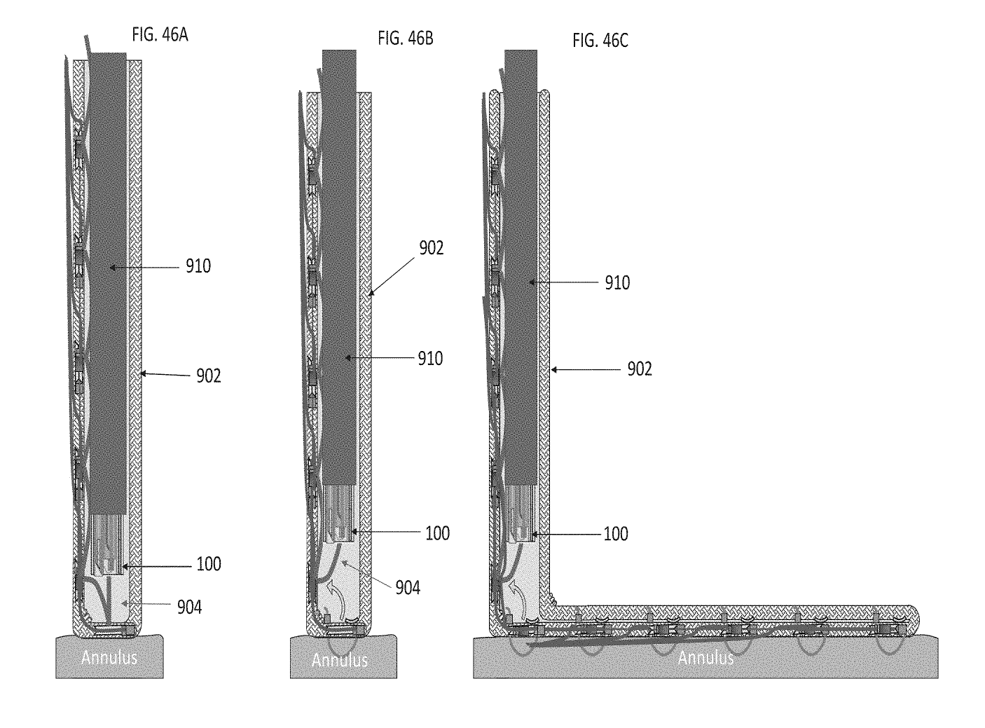

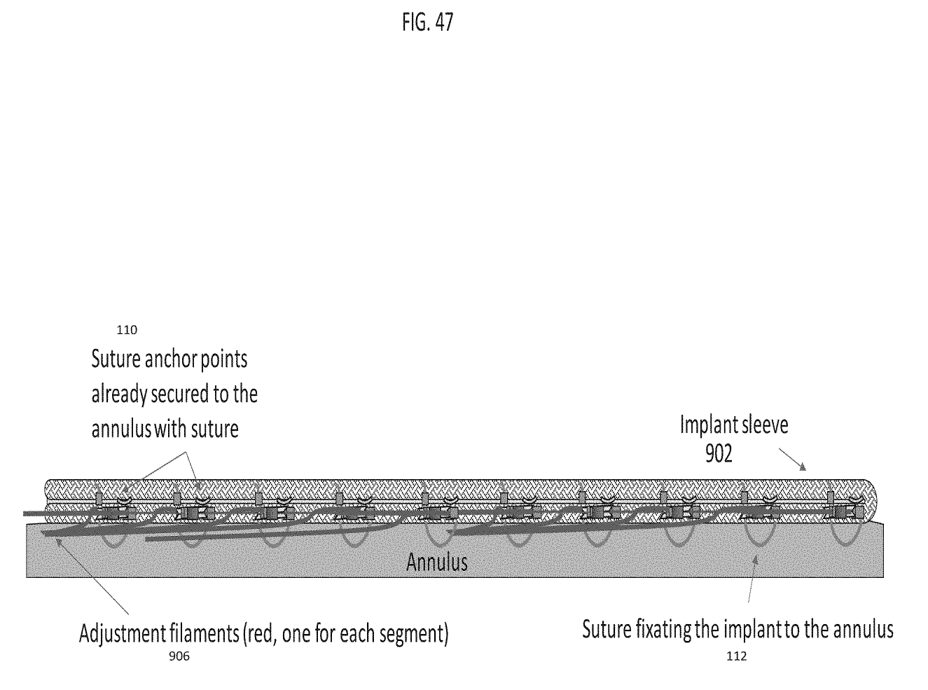

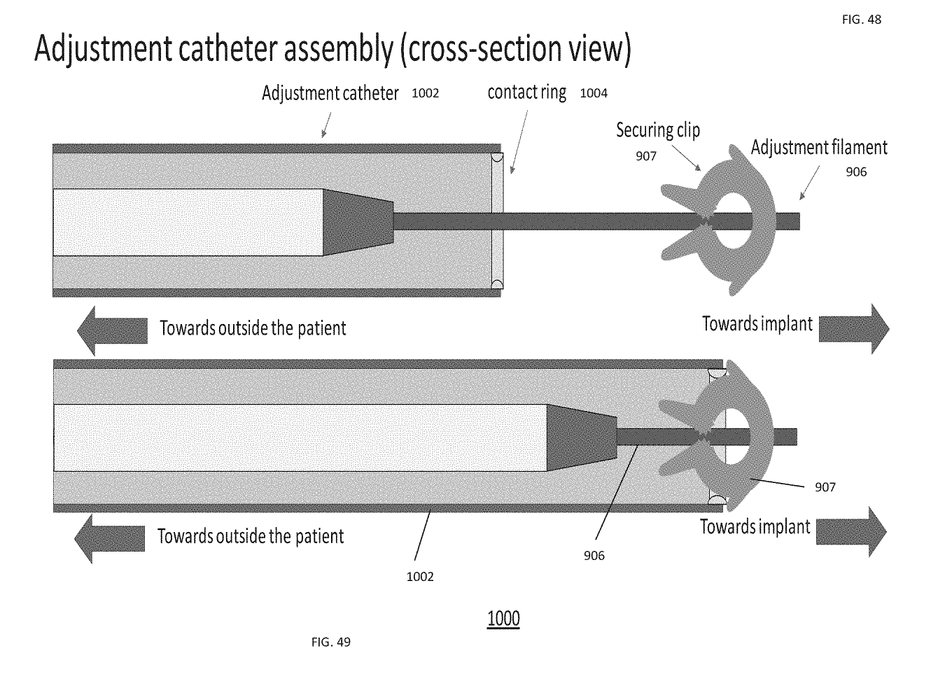

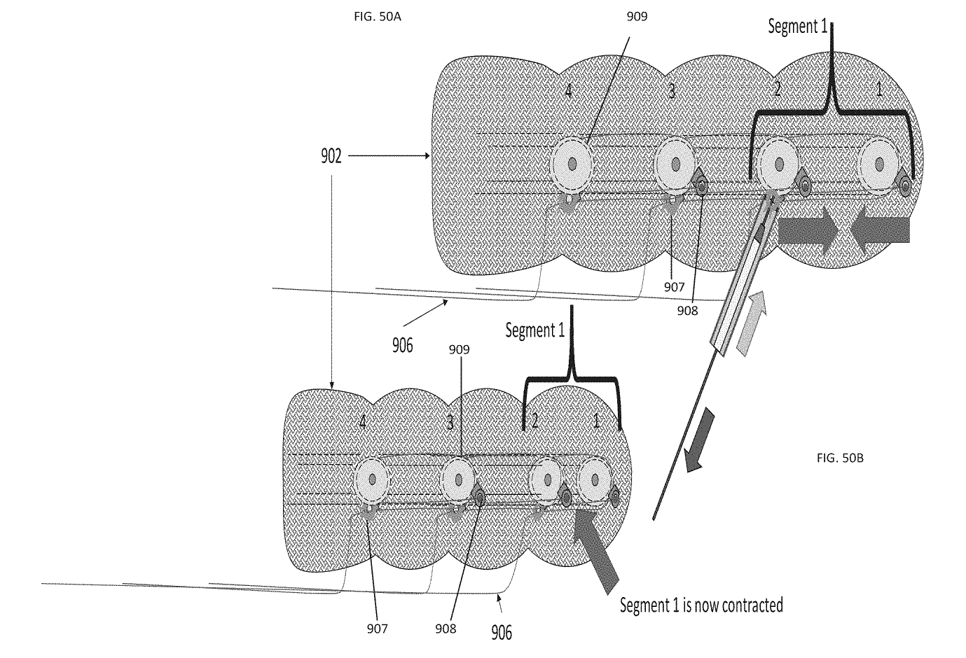

FIGS. 19-41K show a plurality of diagrams of a plurality of embodiments of a continuous driving system for transcatheter/minimally invasive suturing in accordance with this disclosure. In particular, these embodiments comprises two flexible shafts rotating in the same direction. These shafts are connected to a system of rollers and driving pinions encased in two mirror image semicircular rails closely apposed together forming a horseshoe shape. The rails are connected via a torsion spring that bends, allowing the rails to approach each other, decreasing the diameter of the device for easier deployment. A standard ⅜ circle curved suturing needle would travel within the rails, propelled by the needle rollers. The proximal needle rollers are driven by the driving pinions attached to the flexible shafts whereas the distal needle rollers are driven directly by the flexible shafts; with both flexible shafts rotating in the same direction thereby allowing for a continuous cycle of driving the needle. The rationale for this arrangement is that the direction of the proximal rollers needs to be contrary to the distal rollers to allow for continuous unidirectional propulsion of the curved needle. The inner surface of the rails could be smooth enough to minimize friction with the needle and allow easy sling of the needle along the length of the rails. Each rail section also has bases (fixation points) for the rollers and driving pinions. Because the rails are open in their inner aspect the suture is never caught and remains free from tangling during each revolution of the needle. The entire assembly may be mounted on a pivoting base to allow suturing in a wide range of angles and three-dimensional spatial orientations. The torsion spring is positioned along the proximal curvature of the curved rail thereby connecting both rail sections to allow for the bending and contraction of overall dimensions in order facilitate insertion through a smaller diameter catheter. Another mechanism to achieve contraction of the overall diameter may involve connecting both rail sections by a hinge-like structure that would allow the device to be folded, thereby significantly decreasing the diameter of the device.

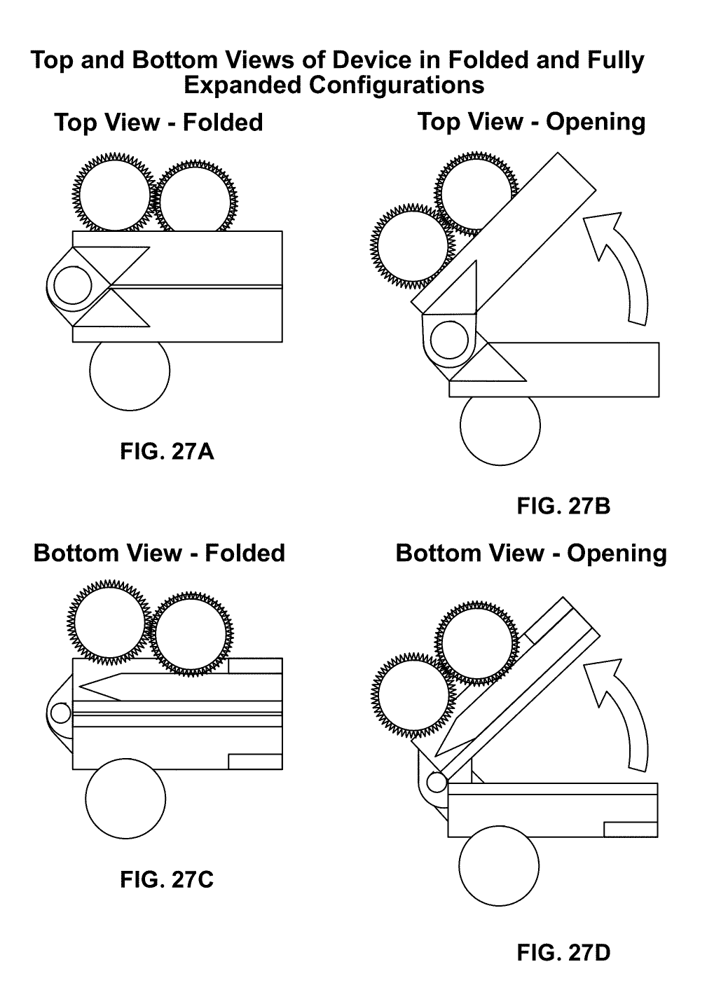

As per FIGS. 19-20, a modified version replaces the torsion spring with a hinge in the center so as to permit folding of the semicircular sections like a book, providing for an even smaller dimension upon insertion through a catheter. Once the suturing system is deployed at the target tissue the hinge would open to orient the semicircular rail sections at 180 degrees to each other, creating a continuous rail structure. Furthermore, the arrangement of the rollers and driving pinion changes in order to allow rotation of the shafts in opposite directions. An additional roller is added in one of the semicircular rail sections to minimize the span between rollers and maximize contact with the needle at all times during the needle-driving cycle. The advantages of this arrangement include elimination of a torque effect that could be caused by rotating both shafts in the same direction and using the opposing torques of the flexible shafts (rotating in opposite directions) to avoid imparting a force upon the hinge that could change the spatial orientation between the semicircular rail sections (i.e. force the hinge to start closing, breaking the continuity between both semicircular rail sections). An additional feature of this embodiment comprises a suturing system in which the entire distal end of the device or portions thereof are capable of rotation and needle positioning in any spatial direction relative to the rest of the device. The entire distal end of the device or portions thereof are also capable of pivoting the needle and/or needle housing structures so as to allow these to point the needle in any given direction or spatial plane without requiring rotation of other portions of the device.

In some embodiments, a transcatheter adjustment is enabled by a possibility to modulate step by step, the ring shape in different mitral annulus segments may play an important role in reducing the degree of mitral regurgitation (MR) and the incidence of recurrent MR overtime. The SATAR is an initial flexible flat ring consisting of a linear assembly of interlocking segments, each of which is comprised of a short central hollow tubular section connecting a proximal and a distal coil wound in opposite (inverted) directions relative to each other. Imagine two corkscrews of opposing pitches placed at 180 degrees from each other and joined in that orientation at their bases by a short hollow tube. These proximal and distal coils are fixed to the central section, meaning that they will not move. A moveable coil having the same diameter and winding direction of its corresponding fixed coil is wound into its corresponding fixed coil on each side (proximal and distal subsegments) of the segment, forming a pair of interwoven coils on each side. These coil pairs are mated together by winding one coil into the other when the moveable coil is turned. The pitch of the coil pairs is inverted on the distal subsegment of each segment compared to the proximal sub-segment. The distal moveable coil of one segment connects to the proximal moveable coil of the next segment by a flexible sleeve having freely rotating junctions connecting it to both coils. There is a suture fixation point at the center of each segment (fixed to the central section) for suture fixation to the annulus. The mechanical principle applied in the SATAR device is the same as in a turnbuckle. In a turnbuckle, you have a central threaded sleeve mated to two screws, one coming in from each end of the sleeve. One screw has a right-hand thread and the other one a left-hand thread. Turning the sleeve in one direction will draw both screws closer together further into the sleeve, shortening the overall length of the turnbuckle. Turning the sleeve in the opposite direction will draw both screws out and farther apart from each other, lengthening the overall length of the turnbuckle. Transcatheter delivery of the SATAR device requires the smallest possible diameter. Therefore, the same turnbuckle principle is applied to the SATAR device design by changing each of the threaded portions of the sleeve and its corresponding screw for coils of the same size that would thread into the empty spaces of each other. The two sides of this “turnbuckle” would join at the center (such as two corkscrews facing in opposite directions joined together at their bases). A very important feature of the SATAR device is that it is delivered as a linear structure that can be inserted through a catheter. A suture fixation point would extend from the central section of each segment. This feature allows for suture fixation of each segment of the SATAR to a different portion of the annular circumference, creating a ring-type structure with the necessary anchor points against which segmental contraction of the SATAR can effect a tailored contraction of different portions of the annulus. Winding of a moveable coil into or out of its corresponding fixed coil is achieved through the engagement of inner ridges projecting towards the lumen of the moveable coil by a flexible shaft traversing the lumen of that moveable coil. Because the SATAR is comprised of coil assemblies and hollow tubular sections a lumen is formed. A flexible drive-shaft with a flattened spatula-like distal end is fed through the lumen of the SATAR and aligned with the inner ridges of the proximal or distal movable coil within each segment. Rotating the flexible drive-shaft in one direction would engage the inner ridges of the moveable coil and rotate that coil accordingly. If, for example, clockwise drive-shaft rotation makes the moveable coil wind itself further into its corresponding fixed coil then the overall length of that coil pair, and therefore that proximal or distal sub-segment, would shorten. It follows that counterclockwise rotation would have the opposite effect, lengthening the overall sub-segment length. To minimize unintended migration of the flexible drive-shaft during SATAR deployment a holding chamber has been included at the distal end of the SATAR. This holding chamber is designed to secure the spatula-like distal end of the flexible drive-shaft during deployment. An intentional simultaneous rotation and pulling of the flexible drive-shaft would be necessary to align the spatula-like distal end of the shaft to the opening of the holding chamber in order to release it into the lumen of the SATAR. The SATAR segments and sub-segments would then be shortened or lengthened as needed from distal to proximal but the flexible drive-shaft can move back and forth within the lumen as needed to re-engage any sub-segment as needed. Because the SATAR is composed of multiple segments, and each segment has proximal and distal sub-segments, significant flexibility is built into the SATAR to allow for tailored segmental (and sub-segmental) annular lengthening or shortening. Such versatility maximizes the capability of the surgeon to optimize the SATAR geometry according to individual patient anatomy in order to achieve the desired reshaping of the mitral valve annulus. Another potential benefit of the SATAR device is that it is comprised of linear segments that could be made from material of variable stiffness so as when contracted the stiffness is increased. The SATAR enables fine-tuning of the ring size and shape after implantation which allows the surgeon to optimize the size and the coaptation length with real-time feedback to improve surgical outcomes. The SATAR is similar in size and shape to standard commercially available rings but can be implanted by a transcatheter approach.

In some embodiments, the SATAR device is transcatheter implanted as follows. The inguinal area (presumably on the right side) is identified and thoroughly cleaned. A surgical field is established. After the surgical field has been prepped and draped as usual the inguinal area is palpated and the femoral vein is punctured using either anatomical landmarks, ultrasound guidance or other means of identification. A guide wire is inserted through the access needle, the needle is removed and the Brockenbrough needle is inserted. After positioning at the superior vena cava, the first movement seen after pulling back the Brockemrough needle is the catheter falling into the right atrium from the SVC and followed by a second more subtle movement as the catheter falls from the thicker muscular intra-atrial septum into the fossa ovalis. The assembly (needle plus guidewire) are then gently advanced and if in the position is correct it may catch on the lip of the fossa. At this point the pressure tracing should demonstrate a higher pressure with a straight line suggesting the tip of the catheter is abutting the intra-atrial septum, thus damping the pressure tracing. After verifying the right position, the septal puncture should be performed and the needle advanced into the transseptal sheath. Once satisfied that the needle is in the LA, a dilator is passed over the wire in preparation for subsequent steps. At this time an introducer is inserted through the venous access port. A steerable sheath catheter containing a tapered tip stylet is inserted through the introducer and navigated under fluoroscopic guidance up the left atrium. From this step onward the remainder of the procedure will be carried out under 3D echocardiography guidance. The suturing device, in its closed (hinged) configuration is loaded with the curved needle and trailing suture thread, leaving only the tip of the needle exposed. The SATAR device is loaded into the suturing device by fitting the suturing device needle tip (already exposed) through the SATAR device's most distal suture anchor slot (anchoring orifice). The loaded suturing device and attached SATAR device are both introduced through the steerable sheath catheter and advanced out of the sheath into the left atrium. Under 3D echo guidance the mitral valve is identified. The anterolateral trigone and anterolateral extreme of the posterior leaflet's P1 segment are identified along with the corresponding segment of the annulus. The suturing device, loaded with suture and the SATAR device, is positioned facing the corresponding segment of the annulus. At this time the suturing system is fully deployed (hinge fully opened). The fully deployed suturing device is apposed against the annulus and the device actuated to drive the suture needle and thread through the annulus. The needle is recaptured into the suturing device and withdrawn from the field along with the suturing device. The SATAR device remains opposed to the annulus but still not fixed to it. Extracorporeally, both ends of the suture thread (identified by different colors for each half of the suture thread) are tied together to form one loop (throw) of the surgical knot. This loop is loaded into spiral knot pusher. The process is repeated with subsequent loops being loaded more proximally along the spiral knot pusher until the desired number of throws is loaded. The loops may be loaded from opposing sides to minimize the occurrence of the loops being disengaged from the knot pusher when exerting tension or going around a curve. Please note that a cutting ring (with internal pegs engaging it to the spiral knot pusher) has already been preloaded on the spiral knot pusher proximal to the last suture loop. The loaded knot pusher is advanced through the introducer, sheath catheter and into the left atrium while maintaining tension on the suture threads. The thread loops will slide along the length of the suture thread as they are pushed by the knot pusher. Once the knot pusher reaches the pierced annular segment it will be turned (rotated) on its axis as to move the knot throws (loops) distally until each loop is delivered under tension to the tissue, forming and tightening the knot. Continued rotation of the knot pusher will drive the cutting ring distally until its cutting edge is forced against the suture threads until they are cut. At this point the first anchoring knot has been delivered and the distal end of the SATAR device is fixed to the annulus. A new suture needle and thread are loaded into the closed (hinged) suturing device as detailed above and the suturing process repeated at each suture anchoring point of the SATAR device. Once the last anchoring suture has been delivered the suture device is withdrawn and the device is ready to effect segmental contraction of the annulus. Under 3D echo guidance the flat head of flexible shaft (residing in the terminal reservoir during implantation) is moved proximally until it engages the inner ridges of the moveable coil belonging to the most distal adjustable segment. Please note that the flexible shaft is marked at is proximal end to allow for its precise displacement and coupling with the inner ridges at different segments. Similarly, the SATAR segments are designed to be identified using 3D echo or fluoroscopy. The flexible shaft is turned, turning the coil and contracting the most distal segment of the SATAR device. This action in turn contracts the corresponding segment of the annulus to which the SATAR is fixed. Once the most distal segment has been adjusted then the flexible shaft is pulled more proximally until the next segment is engaged and contracted as desired. The process is repeated under 3D echo visualization until the desired amount of contraction (or expansion if desired) is attained and a tailored, segmentally optimized annuloplasty has been achieved as per real-time 3D echo parameters. The entire process is carried out with a beating heart and with the possibility of performing immediate adjustments to maximize the efficiency and therapeutic benefit of the procedure.

In some embodiments, a corkscrew knot pusher is employed. This concept is based on the principle of the Archimedean screw. A classical demonstration of this principle is turning the screw within a tube to lift and transfer water out of a river or other body out of water. As the screw turned, the water would be displaced upwards, moving along its continuous spiraling thread until it exited out of the other end. It follows then that if you turn the screw in the opposite direction the water would be displaced downward. The corkscrew knot pusher employs a similar mechanism for pushing the knots through a catheter. By loading the looped suture filaments between the threads of the screw and turning the screw at the end of the corkscrew knot pusher, the loop would be pushed down with every turn of the screw. Because the arch-knot pusher would be opposed to the tissue and would allow the user to keep constant tension on the suture while delivering the knots, all knots could be delivered simultaneously, by simply placing subsequent loops of suture at different levels in the thread of the screw. A sharp-edged (cutting) ring with internal pegs that run along the threads (trailing the last suture loop) can be preloaded allowing for the sutures to be cut immediately after formation of the knot in the same step without additional instruments. Multiple throws can be loaded simultaneously needing only a single step to form the knot while maintaining tension on the suture strands with the knot pusher. A cutting band (ring) can be preloaded so it is positioned trailing the last throw of the knot. Internal pegs wound engage the band with the threads of the screw, being gradually moved distally with every turn as described for the sutures strands. As the band approaches the formed knot the suture strands (still under tension) will be forced against the sharp edge of the band and get cut. This design allows single-step knot pushing under tension and suture cutting.

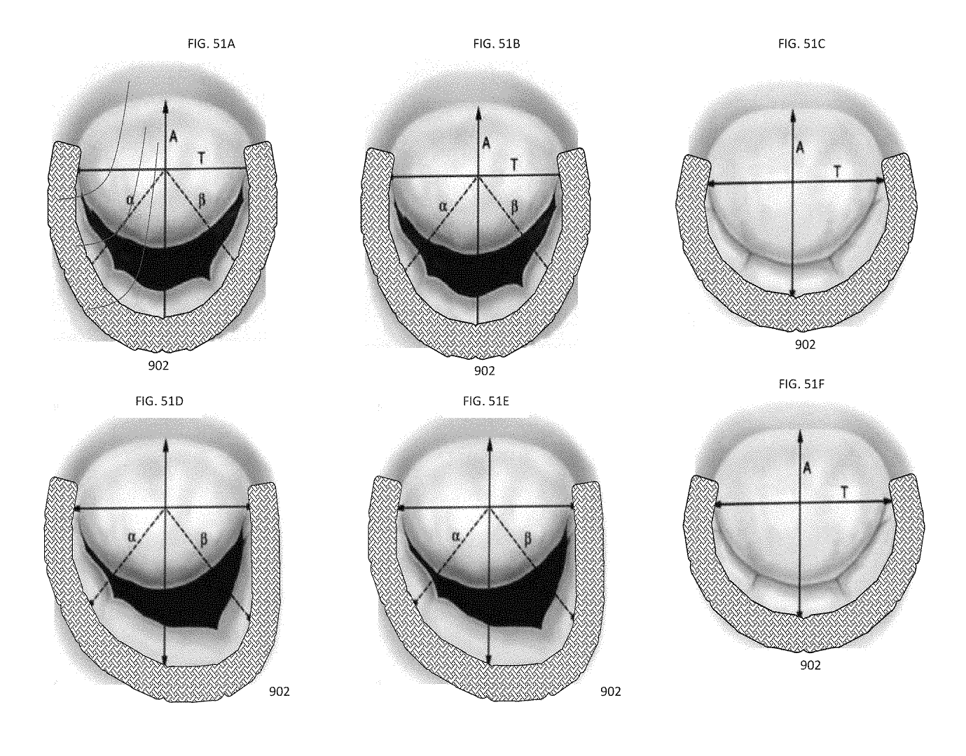

FIGS. 42-43 show a plurality of diagrams of a plurality of embodiments of entering and exiting a threading device and an elongated member in accordance with this disclosure. In particular, a catheter can be used to insert and remove a threading device and an elongated member, as disclosed herein.

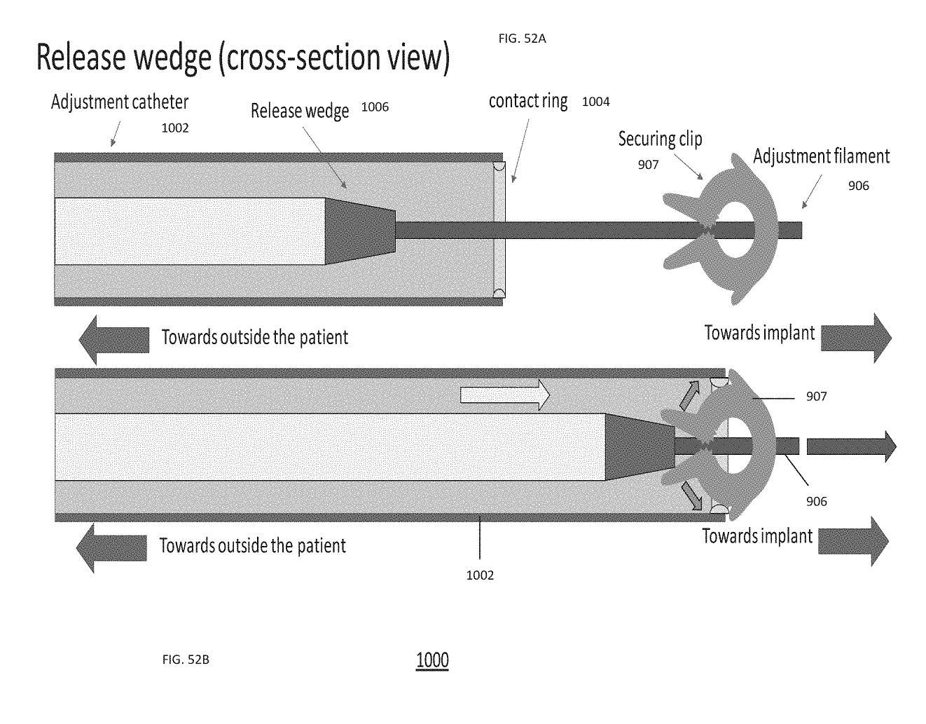

FIGS. 44A-47 show a plurality of diagrams of a plurality of embodiments of an elongated member hosting a threading device in accordance with this disclosure. In particular, a device 900 includes a fabric sleeve 902 and a suturing device guide filament 904. The fabric sleeve 902 includes a closed distal end portion 905 (e.g., tip). The fabric sleeve 902 includes a plurality of segmental adjustment filaments 906, a plurality of cleat cam-like securing clips 907, and a plurality of loop filament anchors 908. The segmental adjustment filaments 906 engage the loop filament anchors 908 such that the fabric sleeve 902 is segmentally adjustable via the cam-like securing clips 907.

The filament 904 can be non-absorbable or absorbable of various gauges. The filament 904 can include silk, cotton, fabric, nylon, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, or other natural or artificial materials. The filament 904 is solid, but can be perforated. The filament 904 is internally dense, but can be hollow. The filament 904 can be rigid, semi-rigid, elastic, resilient, or flexible. For example, the filament 904 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The filament 904 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical.

As shown in FIGS. 44A-44B and 45A-45E, the segmental adjustment mechanisms are tightly sandwiched between two layers of fabric stitched (or otherwise secured) together. An additional fabric layer creates the sleeve 902, closed at its distal end 905 (like a sock). This allows the fabric sleeve 902 to be implanted and mounted over the distal end of the suturing device catheter for simultaneous insertion through a sheath catheter 911 and fixation of the first suture anchor 908 to the annulus. The fabric sleeve 902 slides off the distal end of the suturing device catheter 911 as subsequent suture anchors (e.g., toggles 110, anchors 908) are fixed to the annulus until the entire implant is deployed and fixed. The guide filament (blue) 904 is threaded through a suture device 910 to guide the suture device 910 to the intended suture anchor site as it is advanced. The suture device 910 includes the threading device 100. Once the anchor site is fixed, the guide filament 904 is gently pulled and dislodged from that anchor site (held by a tab or similar) while remaining tethered to the other anchor sites. The process is repeated until the guide filament 904 is dislodged from the last anchor site after its fixation and the filament is withdrawn.

As shown in FIG. 46A, the guide filament 904 is threaded through the suturing device catheter before insertion and suturing device 910 is advanced over the guide filament 904 until the first anchor site is reached. Note that as the fabric sleeve 902 is pressed against the annulus, the suturing device 910 approaches the first anchor site and slight tension is placed on the guide filament 904 and the orientation of the anchor site changes.

As shown in FIG. 46B, the first suture anchor has been secured to the annulus (pink). The suturing device 910 is gently retracted. The guide filament 904 is gently pulled and dislodged from the holding tab at the suture anchor site.

As shown in FIG. 46C, as additional suture anchors are fixed to the annulus, the fabric sleeve 902 progressively slides off the suturing device 910. This process is repeated until the entire fabric sleeve 902 is fixed and the suturing device 910 is withdrawn (e.g., telescoping). The guide filament 904 is also progressively pulled and dislodged from its holding tab on the suture anchors as the suture anchors are fixed until the guide filament 902 is dislodged from the last suture anchor and removed.

As shown in FIG. 47, the fabric sleeve 902 is implanted to the annulus. The suturing device 910 and its guide filament 904 have now been removed. Note that although the fabric sleeve 902 has been depicted as being fixed in a straight linear configuration this is done for illustrative purposes only. For anatomical correctness, it must be remembered that the mitral valve annulus is D-shaped and the fabric sleeve 902 will be fixed to the posterior portion of the annulus corresponding to the curved portion of the D. Now that the fabric sleeve 902 is fixed, the next step is proceeding with the selective contraction of segments as desired to achieve both contraction and reshaping of the annulus. The physician (or another service provider) performing the procedure can decide the number of segments to be contracted, the magnitude of the contraction at each segment and the sequence of contraction. Because the segmental contraction (adjustment) mechanism is tension-based and is reversible, the physician may also decide to release the tension on any segment in a controlled fashion. This is achieved with the aid of the adjustment catheter that is here disclosed herein. Once the desired configuration and degree of contraction throughout the fabric sleeve 902 has been achieved, the adjustment filaments can be cut using the cutter feature (e.g., the cutting edge 120) included in the adjustment catheter.

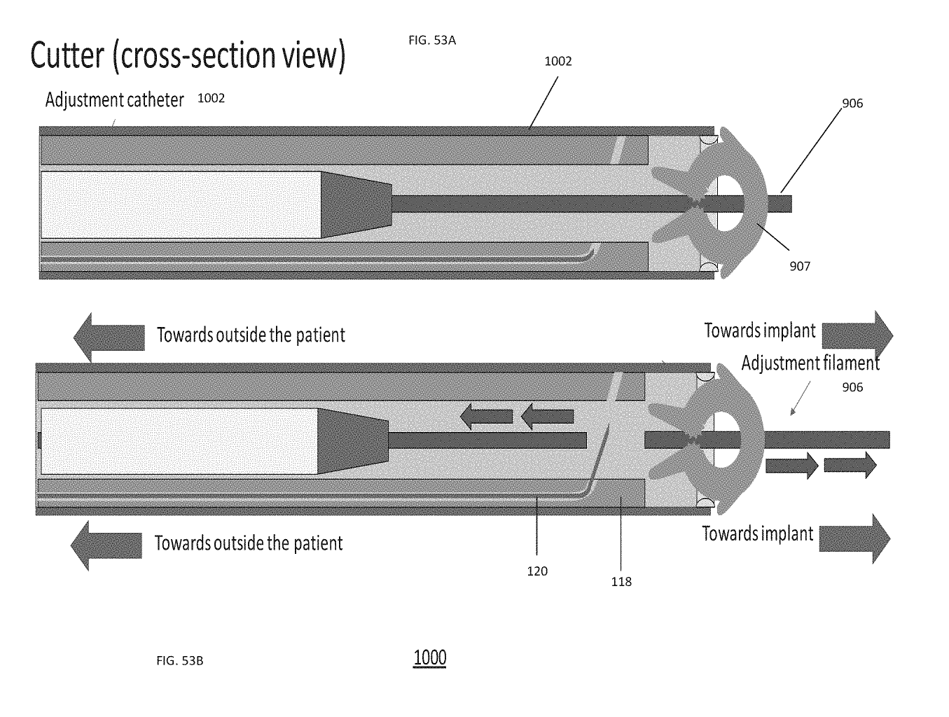

FIGS. 48-52B show a plurality of diagrams of a plurality of embodiments of a technique of adjusting an elongated member in accordance with this disclosure. In particular, a system 1000 is used for adjusting an elongated member, such as the elongated member 9000. As shown in FIGS. 48-49, after the elongated member (e.g., the sleeve 902) has been secured, at least some segmental adjustment is carried out to effect the desired reduction and reshaping of the mitral annulus. Each adjustment filament 906 that comes out of the implant's segmental adjustment mechanism is held tightly at its initial position by a securing clip 907, as shown in FIG. 48. This clip 907 is attached to the proximal suture anchor point within its corresponding segment. To contract the segment, the adjustment filament 906 is threaded through an adjustment catheter 1002 and a release wedge 1006, which travels inside the adjustment catheter 1002. The adjustment catheter 1002 is advanced until the adjustment catheter 1002 engages the securing clip 907, as shown in FIG. 49. A rigid contact ring 1004 at the end of the adjustment catheter 1002 provides a more stable contact area between the adjustment catheter 1002 and the clip 907. The rigid contact ring 1004 can be unitary or assembled with the adjustment catheter 1002. The rigid contact ring 1004 can include plastic, metal, rubber, or others.

As shown in FIGS. 50A-50B, the adjustment catheter 1002 is engaging the securing clip 907 at suture anchor 2 (e.g., the anchor 908), which is the proximal anchor 908 of segment 1. The adjustment catheter 1002 has been cut short for visualization of the adjustment filament 906. The adjustment catheter 1002 is pushed towards the securing clip 907, while the adjustment filament 906 is pulled, shrinking a loop between suture anchors 1 and 2 and contracting segment 1. This process is repeated in as many segments as needed and to the extent desired to achieve optimal reduction and reshaping of the annulus. If for any reason any segment needs to be re-expanded, then the release wedge 1006 can be advanced through the adjustment catheter 1002 to open the securing clip and release the tension from the filament loop. Note that FIGS. 51A-51F show the elongated device 900 being adjusted relative to the annulus of the mitral valve based on various adjustments, as disclosed herein.

As shown in FIGS. 52A-52B, if, at any time during or after adjustment of any segment, at least some tension needs to be relieved from any segment to allow its re-expansion, then the release wedge 1006 is advanced through the adjustment catheter 1002, while the adjustment catheter 1002 is engaging the clip 907. This action opens up the clip 907, thereby releasing tension from the adjustment filament 906 and loosening the filament loop 906 of the intended segment permitting its re-expansion or adjustment.

As shown in FIGS. 53A-53B, at the end of the procedure, after all final adjustments have been made, the adjustment filaments 906 can be cut using the cutter (e.g., the cutting edge 120) included in the adjustment catheter 1002. The adjustment filament 906 to be cut is held under tension and the cutter is advanced. The cut filament can now be removed. Note that the release wedge 1006 can travel parallel to the cutter or toward or away from the cutter.

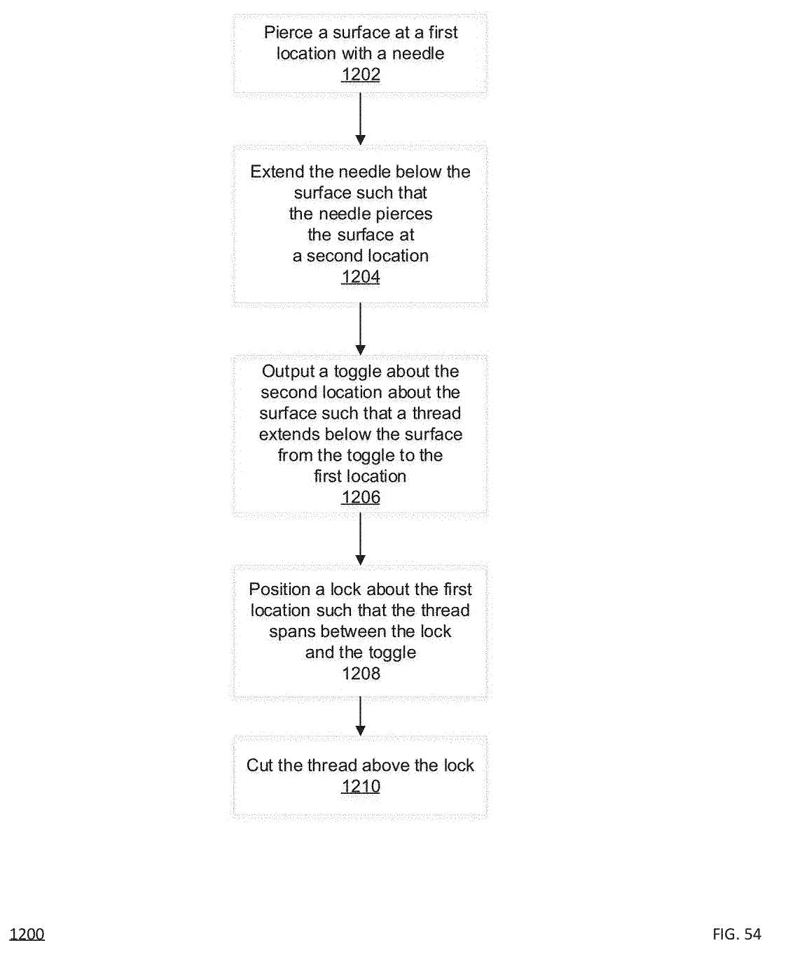

FIG. 54 shows a flowchart of a process for threading in accordance with this disclosure. In particular, a process 1200 includes a plurality of blocks 1202-1210, which are performed, as explained in FIGS. 1-53.

In block 1202, a surface (e.g., the layer 126 or the volume 128) is pierced at a first location with a needle (e.g., the needle 106). The needle includes a toggle (e.g., the toggle 110) and a thread (e.g., the thread 112). The toggle is coupled to the thread. The surface can be an inanimate surface. The surface can be an animate surface. Block 1202 is also shown in FIGS. 1-5B.

In block 1204, the needle is extended below the surface such that the needle pierces the surface at a second location. The needle can form a C-shape or U-shape or an arc or a crescent when extending below the surface and piercing the second location. The needle can include a shape memory material. Block 1204 is also shown in FIGS. 1-5B

In block 1206, the toggle is output about the second location above the surface such that the thread extends below the surface from the toggle to the first location. Block 1206 is also shown in FIGS. 1-5B

In block 1208, a lock (e.g., the lock 124) is positioned about the first location such that the thread spans between the lock and the toggle. Block 1208 is also shown in FIGS. 1-5B

In block 1210, the thread is cut above the lock (e.g., via the cutting edge 120). The thread can be extended through a sleeve (e.g., the elongated member 902) between the toggle and the lock. Block 1210 is also shown in FIGS. 1-5B

The sleeve can include a plurality of segments and the segments can be adjusted independent of each other from outside the sleeve. The segments can be adjusted independent of each other from outside the sleeve based on a puppeteering mode of control.

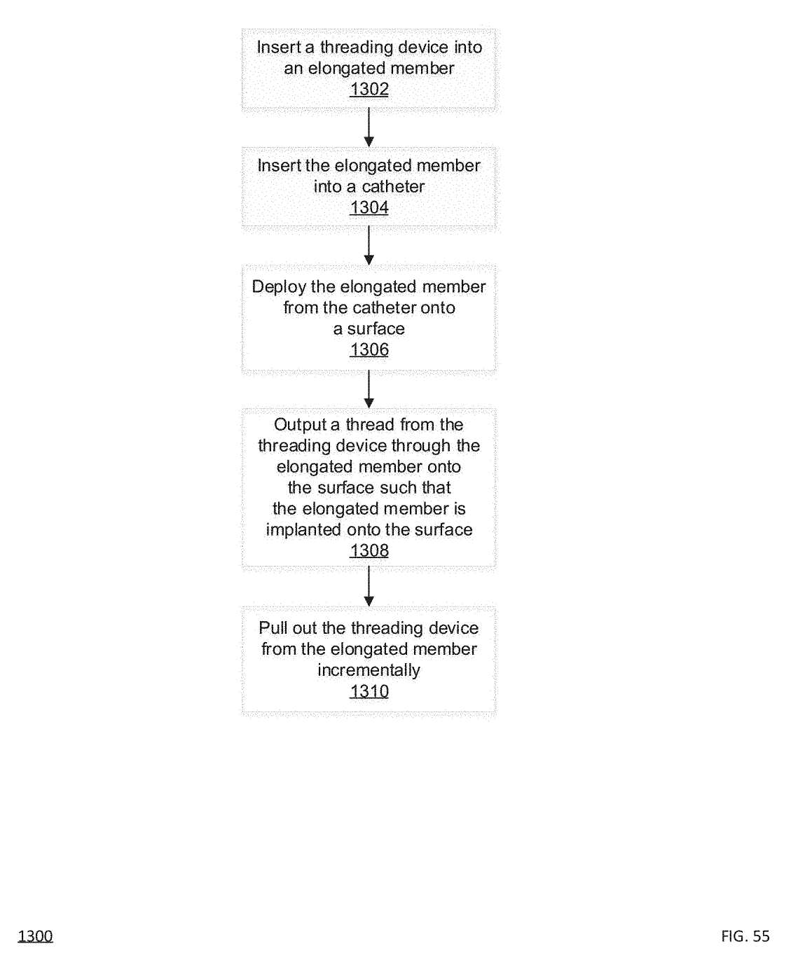

FIG. 55 shows a flowchart of a process for coupling an elongated member to a surface in accordance with this disclosure. In particular, a process 1300 includes a plurality of blocks 1302-1310, which are performed, as explained in FIGS. 1-54.

In block 1302, a threading device (e.g., threading device 100) is inserted into an elongated member (e.g., sleeve 902). The elongated member is mounted over the threading device. The elongated member has a guide filament. The guide filament is threaded through the threading device. This is also shown in FIGS. 44A-47.

In block 1304, the elongated member, with the threading device, is inserted into a catheter (e.g., sheath catheter 911). This is also shown in FIGS. 44A-47.

In block 1306, the elongated member is deployed (e.g., telescoped out) from the catheter onto a surface (e.g., animate surface, inanimate surface). This is also shown in FIGS. 44A-47.

In block 1308, the threading device outputs a thread (e.g., thread 112) through the elongated member onto the surface such that the elongated member is implanted (e.g., threaded) onto the surface. This can be done via the process 1200. This is also shown in FIGS. 44A-47.

In block 1310, the threading device is pulled out from the elongated member incrementally, as shown in FIG. 46C. Since the elongated member has an inner cavity and an end portion, where the threading device is hosted in the cavity before the threading device is pulled out, as the threading device is pulled out of the inner cavity away from the end portion, the inner cavity may collapse or reduce in volume or decrease in height or reduce in vertical thickness. This is also shown in FIGS. 44A-47. However, not that this is illustrative. For example, the elongated member can be secured or coupled to the surface without piercing the surface, such as, for example, using magnets, suction cups, adhesives, glues, hook and loop fasteners, snaps, thermal bonding, light energy, heat energy, and/or chemical bonding. The segments can be adjustable independent of each other when the sleeve is secured to the surface without piercing the surface.

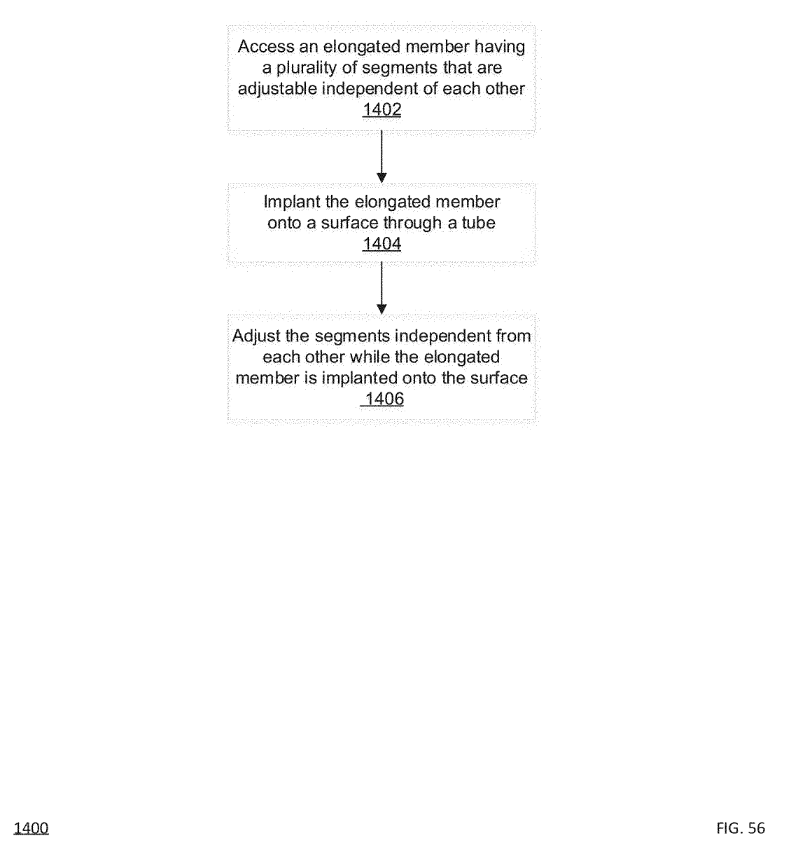

FIG. 56 shows a flowchart of a process for adjusting an elongated member in accordance with this disclosure. In particular, a process 1400 includes a plurality of blocks 1402-1410, which are performed, as explained in FIGS. 1-55.

In block 1402, a user (e.g., physician) accesses an elongated member (e.g., sleeve 902) having a plurality of segments that are adjustable independent of each other, whether external or internal to the elongated member. This is also shown in FIGS. 8A-13.

In block 1404, the user implants (e.g., sutures) the elongated member onto a surface (e.g., animate, inanimate) through a tube (e.g., catheter). This is also shown in FIGS. 44A-47.

In block 1406, the user adjusts (e.g., filaments, ratchets, spools) the segments independent of each other, while the elongated member is implanted onto the surface. This is also shown in FIGS. 8A-13. The adjustment can also take place via the clips 907, as shown in FIGS. 48-53.

As such, in some embodiments, based on FIGS. 1-55, a device can include a sleeve (e.g., 902) including a plurality of segments that are adjustable independent of each other when the sleeve is secured onto a surface. The surface can be an inanimate surface. The inanimate surface can be configured for threading therethrough from within the sleeve (e.g., via the threading device 100). The surface can be an animate surface. The animate surface can be a tissue. The tissue can be an organ tissue. The organ tissue can be a heart tissue. The segments can be adjustable independent of each other when the sleeve is secured onto the surface from within the sleeve not through a plurality of anchors (e.g., via the threading device 100). The segments can be adjustable independent of each other when the sleeve is secured onto the surface from within the sleeve via a plurality of sutures (e.g., via the threading device 100). The sleeve can contain a catheter (e.g., the threading device 100). The heart tissue can be a valve tissue. The valve tissue can be a mitral valve having an annulus, where the annulus has a size and a shape, where the segments are adjustable independent of each other when the sleeve is secured onto the surface from within the sleeve such that the sleeve reduces the size and preserves the shape. The valve tissue can be a tricuspid valve. The sleeve can contain a tube (e.g., the threading device 100). The tube can contain a cutting edge (e.g., the cutting edge 120), a thread (e.g., the thread 112), and a retractable foot (e.g., the foot 116). The surface can be a tissue of a heart valve. The tube can contain a needle (e.g., the needle 106), a toggle (e.g., the toggle 110), and a thread (e.g., the thread 112), where the toggle is coupled to the thread, where the needle carries the toggle and the thread. The surface can be a tissue of a heart valve. The sleeve can contain a needle (e.g., the needle 106) that is hollow, where the needle includes a shape memory material. The shape memory material can have a default non-rectilinear shape. The default non-rectilinear shape can include an arcuate end portion. The tube is removable from the sleeve. The sleeve includes a closed end portion. The segments are adjustable independent of each other when the sleeve is secured onto the surface through a plurality of threads. The sleeve can be an annuloplasty device (e.g., the elongated member 902). The segments can be adjustable independent of each other based on the filaments configured for adjustment independent of each other. The filaments can be configured for adjustment independent of each other based on a plurality of clips (e.g., clips 312) that are configured for adjustment independent of each other, where the filaments are tensioned based on the clips. The clips are permanently coupled to the sleeve. The sleeve can includes a plurality of anchors (e.g., anchors 406) and a plurality of cords (e.g., the chords 408), where the cords span between the anchors, where the segments are adjustable independent of each other when the sleeve is secured onto the surface based on the anchors enabling the cords to be independently wedged thereto, where each of the anchors hosts at least two of the cords. At least two of the cords can longitudinally extend along at least two different longitudinal planes that are laterally spaced apart from each other. The sleeve includes a plurality of anchors (e.g., anchors 506) and a plurality of ratchet strips (e.g., J-shaped bars 508), where the ratchet strips span between the anchors, where the segments are adjustable independent of each other when the sleeve is secured onto the surface based on the anchors enabling the ratchet strips to be independently moved. At least one of the anchors can include a portion that is configured for releasing at least one of the strips such that the at least one of the strips is bi-directionally movable. The sleeve can include a plurality of tabs (e.g., tension tabs 616) and a plurality of filaments (e.g., the tension sutures 612), where the filaments extend through the tabs such that the filaments run interior to the sleeve and exterior to the sleeve, where the segments are adjustable independent of each other when the sleeve is secured onto the surface based on the tabs enabling the filaments to be independently tensioned. The filaments can loop about the tabs such that the tabs operate as a plurality of pulleys. The surface is a first surface, where the sleeve includes a plurality of filaments s (e.g., the lasso-style filaments 710) woven thereinto, where the filaments include a plurality of longitudinal end portions coupled to a winch (e.g., the spool 704), where the winch is coupled to a second surface, where the filaments are tensioned via the winch. The first surface can be a tissue of a heart valve. The winch can include a spool coupled to the filaments, where the spool includes a cavity, wherein the cavity hosts a guidewire (e.g., the guidewire 702). The winch can have a bidirectional locking and unlocking mechanism.

As such, in some embodiments, based on FIGS. 1-55, a device can include a first tube (e.g., the first tube 102), a second tube (e.g. the second tube 104) extending within the first tube, a needle (e.g., the needle 106) including a shape memory material, where the needle is switchable between a non-default shape and a default shape based on the shape memory material, where the needle has the non-default shape when the needle extends within the second tube, where the needle has the default shape when the needle extends outside the second tube, a rod (e.g., the rod 106) extending within the needle, a toggle (e.g., the toggle 110) extending within the needle, where the rod engages the toggle, a thread (e.g., the thread 112) extending within the first tube, where the thread is coupled to the toggle, a lock (e.g., the lock 124) extending within the first tube, where the lock is coupled to the thread, a foot (e.g., the foot 116) extending within the first tube, where the foot is retractable out of the first tube, and a cutting edge (e.g., the cutting edge 120) extending within the first tube, where the cutting edge is sufficiently sharp to cut the thread above the lock. The device can include a third tube (e.g., the third tube 118) extending within the first tube between the second tube and the foot, where the cutting edge extends within the third tube. The thread can extend through the third tube. The needle can be configured to puncture a surface (e.g., the layer 126 or the volume 128), where the surface is an inanimate surface through which the thread can be threaded. The needle is configured to puncture a surface (e.g., the layer 126 or the volume 128), where the surface is an animate surface through which the thread can be threaded. The animate surface can be of a heart. The needle is configured to puncture a surface (e.g., the layer 126 or the volume 128), where the surface is an inanimate surface through which the thread can be threaded. The needle can be configured to puncture a surface (e.g., the layer 126 or the volume 128), where the surface is an animate surface through which the thread can be threaded. The animate surface can be a tissue. The tissue can be an organ tissue. The organ tissue can be a heart tissue. The foot can retract out of the first tube towards the needle in the default shape. The first tube can extends within a sleeve (e.g., the elongated member 902). The sleeve can include a plurality of segments that are adjustable independent of each other when the sleeve is positioned on a surface (e.g., the layer 126 or the volume 128). The needle can be configured to puncture the surface, where the surface is an inanimate surface through which the thread can be threaded from within the sleeve. The needle can be configured to puncture the surface, where the surface is an animate surface through which the thread can be threaded from within the sleeve. The animate surface can be a tissue. The tissue can be an organ tissue, a heart tissue, or a valve tissue. The valve tissue can be of at least one of a mitral valve or a tricuspid valve. The valve tissue can be the mitral valve, where the mitral valve has an annulus, where the annulus has a size and a shape, where the segments are adjustable independent of each other when the sleeve is secured onto the surface from within the sleeve such that the sleeve reduces the size and preserves the shape. The segments can be adjustable independent of each other when the sleeve is secured onto the surface from within the sleeve not through a plurality of anchors. The segments can be adjustable independent of each other when the sleeve is secured onto the surface from within the sleeve via the thread. The sleeve can include a closed end portion that faces the first tube. The sleeve can be an annuloplasty device (e.g., the elongated member 902). The needle can have an end portion including the shape memory material, where the end portion is non-rectilinear in the default shape. The end portion can include an arcuate portion. The second tube can be telescoping relative to the first tube.

As such, in some embodiments, based on FIGS. 1-55, a method can include laying a sleeve (e.g., the elongated member 902) onto a surface (e.g., the annulus), where the sleeve includes a plurality of segments, securing the sleeve onto the surface from within the sleeve (e.g., via the threading device 100); and adjusting the segments independent of each other from outside the sleeve (e.g., via the segmental adjustment filament 906). The segments can be adjusted independent of each other from outside the sleeve based on the sleeve not being anchored to the surface. The segments can be adjusted independent of each other from outside the sleeve based on the sleeve being threaded to the surface from within the sleeve. The segments can be adjusted independent of each other from outside the sleeve based on a puppeteering mode of control. The surface can be an inanimate surface. The surface can be an animate surface. The sleeve can be secured onto the surface from a tube extending within the sleeve removably. The tube can be a catheter. The catheter can include a needle (e.g., the needle 106), a rod (e.g., the rod 108), a toggle (e.g., the toggle 110), a thread (e.g., the thread 112), a lock (e.g., the lock 124), and a cutting edge (e.g., the cutting edge 120), where the needle includes a shape memory material, where the rod extends within the needle, where the toggle extends within the needle, where the rod engages the toggle, where the thread is coupled to the toggle, where the lock is coupled to the thread, where the cutting edge is sufficiently sharp to cut the thread above the lock.

As such, in some embodiments, based on FIGS. 1-55, a method can include piercing a surface (e.g., the layer 126 or the volume 128) at a first location with a needle (e.g., the needle 106), where the needle includes a toggle (e.g. the toggle 110) and a thread (e.g., the thread 112), where the toggle is coupled to the thread, extending the needle below the surface such that the needle pierces the surface at a second location, outputting the toggle about the second location above the surface such that the thread extends below the surface from the toggle to the first location, positioning a lock (e.g., the lock 124) about the first location such that the thread spans between the lock and the toggle, and cutting the thread above the lock (e.g., via the cutting edge 120). The surface can be an inanimate surface. The surface can be an inanimate surface. The thread can be extended through a sleeve (e.g., the elongated member 902) between the toggle and the lock. The sleeve can include a plurality of segments and the segments can be adjusted independent of each other from outside the sleeve. The segments can be adjusted independent of each other from outside the sleeve based on a puppeteering mode of control.

As such, in some embodiments, based on FIGS. 1-55, a device can include an elongated member (e.g., the elongated member 902) including a plurality of segments that are adjustable independent of each other when the elongated member is secured onto a surface of a valve of a heart. At least one of the segments is helical.

Features described with respect to certain embodiments may be combined in or with various some embodiments in any permutational or combinatory manner. Different aspects or elements of example embodiments, as disclosed herein, may be combined in a similar manner.

Although the terms first, second, can be used herein to describe various elements, components, regions, layers, or sections, these elements, components, regions, layers, or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Features described with respect to certain example embodiments can be combined and sub-combined in or with various other example embodiments. Also, different aspects or elements of example embodiments, as disclosed herein, can be combined and sub-combined in a similar manner as well. Further, some example embodiments, whether individually or collectively, can be components of a larger system, wherein other procedures can take precedence over or otherwise modify their application. Additionally, a number of steps can be required before, after, or concurrently with example embodiments, as disclosed herein. Note that any or all methods or processes, at least as disclosed herein, can be at least partially performed via at least one entity in any manner.

Example embodiments of this disclosure are described herein with reference to illustrations of idealized embodiments (and intermediate structures) of this disclosure. As such, variations from various illustrated shapes as a result, for example, of manufacturing techniques or tolerances, are to be expected. Thus, various example embodiments of this disclosure should not be construed as necessarily limited to various particular shapes of regions illustrated herein, but are to include deviations in shapes that result, for example, from manufacturing.

Any or all elements, as disclosed herein, can be formed from a same, structurally continuous piece, such as being unitary, or be separately manufactured or connected, such as being an assembly or modules. Any or all elements, as disclosed herein, can be manufactured via any manufacturing processes, whether additive manufacturing, subtractive manufacturing, or other any other types of manufacturing. For example, some manufacturing processes include three dimensional (3D) printing, laser cutting, computer numerical control routing, milling, pressing, stamping, vacuum forming, hydroforming, injection molding, lithography, and so forth.

Various corresponding structures, materials, acts, and equivalents of all means or step plus function elements in various claims below are intended to include any structure, material, or act for performing the function in combination with other claimed elements as specifically claimed. Various embodiments were chosen and described in order to best explain various principles of this disclosure and various practical applications thereof, and to enable others of ordinary skill in a pertinent art to understand this disclosure for various embodiments with various modifications as are suited to a particular use contemplated.

This detailed description has been presented for various purposes of illustration and description, but is not intended to be fully exhaustive or limited to this disclosure in various forms disclosed. Many modifications and variations in techniques and structures will be apparent to those of ordinary skill in an art without departing from a scope and spirit of this disclosure as set forth in various claims that follow. Accordingly, such modifications and variations are contemplated as being a part of this disclosure. Scope of this disclosure is defined by various claims, which include known equivalents and unforeseeable equivalents at a time of filing of this disclosure.

1. A device comprising:
a sleeve including a plurality of segments that are adjustable independent of each other when the sleeve is secured to a surface.

2. The device of claim 1, wherein the surface is an inanimate surface.

3. The device of claim 2, wherein the inanimate surface is configured for threading therethrough from within the sleeve.

4. The device of claim 1, wherein the surface is an animate surface.

5. The device of claim 4, wherein the animate surface is a tissue.

6. The device of claim 5, wherein the tissue is an organ tissue.

7. The device of claim 6, wherein the organ tissue is a heart tissue.

8. The device of claim 1, wherein the segments are adjustable independent of each other when the sleeve is secured to the surface without piercing the surface.

9. The device of claim 1, wherein the segments are adjustable independent of each other when the sleeve is secured to the surface from within the sleeve via a plurality of sutures.

10. The device of claim 1, wherein the sleeve contains a catheter.

11. The device of claim 7, wherein the heart tissue is a valve tissue.

12. The device of claim 11, wherein the valve tissue is a mitral valve having an annulus, wherein the annulus has a size and a shape, wherein the segments are adjustable independent of each other when the sleeve is secured onto the surface from within the sleeve such that the sleeve reduces the size and preserves the shape.

13. The device of claim 11, wherein the valve tissue is a tricuspid valve.

14. The device of claim 1, wherein the sleeve contains a tube.

15. The device of claim 14, wherein the tube contains a cutting edge, a thread, and a retractable foot.

16. The device of claim 1, wherein the surface is a tissue of a heart valve.

17. The device of claim 14, wherein the tube contains a needle, a toggle, and a thread, wherein the toggle is coupled to the thread, wherein the needle carries the toggle and the thread.

18. The device of claim 17, wherein the surface is a tissue of a heart valve.

19. The device of claim 1, wherein the sleeve contains a needle that is hollow, wherein the needle includes a shape memory material.

20. The device of claim 19, wherein the shape memory material has a default non-rectilinear shape.

21. The device of claim 20, wherein the default non-rectilinear shape includes an arcuate end portion.

22. The device of claim 14, wherein the tube is removable from the sleeve.

23. The device of claim 1, wherein the sleeve includes a closed end portion.

24. The device of claim 1, wherein the segments are adjustable independent of each other when the sleeve is secured to the surface through piercing the surface.

25. The device of claim 24, wherein the sleeve is an annuloplasty device.

26. The device of claim 1, wherein the segments are adjustable independent of each other based on a plurality of filaments configured for adjustment independent of each other, wherein the filaments are coupled to the segments.

27. The device of claim 26, wherein the filaments are configured for adjustment independent of each other based on a plurality of clips that are configured for adjustment independent of each other, wherein the filaments are tensioned based on the clips.

28. The device of claim 27, wherein the clips are permanently coupled to the sleeve.

29. The device of claim 1, wherein the sleeve includes a plurality of anchors and a plurality of cords, wherein the cords span between the anchors, wherein the segments are adjustable independent of each other when the sleeve is secured onto the surface based on the anchors enabling the cords to be independently wedged thereto, wherein each of the anchors hosts at least two of the cords.

30. The device of claim 29, wherein the at least two of the cords longitudinally extend along at least two different longitudinal planes that are laterally spaced apart from each other.

31. The device of claim 1, wherein the sleeve includes a plurality of anchors and a plurality of ratchet strips, wherein the ratchet strips span between the anchors, wherein the segments are adjustable independent of each other when the sleeve is secured onto the surface based on the anchors enabling the ratchet strips to be independently moved.

32. The device of claim 31, wherein at least one of the anchors includes a portion that is configured for releasing at least one of the strips such that the at least one of the strips is bi-directionally movable.

33. The device of claim 1, wherein the sleeve includes a plurality of tabs and a plurality of filaments, wherein the threads extend through the tabs such that the filaments run interior to the sleeve and exterior to the sleeve, wherein the segments are adjustable independent of each other when the sleeve is secured onto the surface based on the tabs enabling the filaments to be independently tensioned.

34. The device of claim 33, wherein the threads loop about the tabs such that the tabs operate as a plurality of pulleys.

35. The device of claim 1, wherein the surface is a first surface, wherein the sleeve includes a plurality of filaments woven thereinto, wherein the filaments include a plurality of longitudinal end portions coupled to a winch, wherein the winch is coupled to a second surface, wherein the filaments are tensioned via the winch.

36. The device of claim 35, wherein the first surface is a tissue of a heart valve.

37. The device of claim 35, wherein the winch includes a spool coupled to the filaments, wherein the spool includes a cavity, wherein the cavity hosts a guidewire.

38. The device of claim 35, wherein the winch has a bidirectional locking and unlocking mechanism.

39. A device comprising:
a first tube;
a second tube extending within the first tube;
a needle including a shape memory material, wherein the needle is switchable between a non-default shape and a default shape based on the shape memory material, wherein the needle has the non-default shape when the needle extends within the second tube, wherein the needle has the default shape when the needle extends outside the second tube;
a rod extending within the needle;
a toggle extending within the needle, wherein the rod engages the toggle;
a thread extending within the first tube, wherein the thread is coupled to the toggle;
a lock extending within the first tube, wherein the lock is coupled to the thread;
a foot extending within the first tube, wherein the foot is retractable out of the first tube; and
a cutting edge extending within the first tube, wherein the cutting edge is sufficiently sharp to cut the thread above the lock.

40-67. (canceled)

68. A method comprising:
laying a sleeve onto a surface, wherein the sleeve includes a plurality of segments;
securing the sleeve to the surface; and
adjusting the segments independent of each other from outside the sleeve.

69-77. (canceled)

78. A method comprising:
piercing a surface at a first location with a needle, wherein the needle includes a toggle and a thread, wherein the toggle is coupled to the thread;
extending the needle below the surface such that the needle pierces the surface at a second location;
outputting the toggle about the second location above the surface such that the thread extends below the surface from the toggle to the first location;
positioning a lock about the first location such that the thread spans between the lock and the toggle; and
cutting the thread above the lock.

79-83. (canceled)

84. A device comprising:
an elongated member including a plurality of segments that are adjustable independent of each other when the elongated member is secured onto a surface of a valve of a heart.

85. (canceled)
